(19) United States
(12) Patent Application Publication
(10) Pub. No.: US 2025/0177452 A1
(43) Pub. Date: Jun. 5, 2025

(54) USE OF IMMUNOSUPPRESSION TO ENABLE ENGRAFTMENT OF HEMATOPOIETIC STEM CELLS

(71) Applicant: The Board of Trustees of the Leland Stanford Junior University, Stanford, CA (US)

(72) Inventors: Agnieszka Czechowicz, Stanford, CA (US); Yan Yi Chan, Sunnyvale, CA (US)

(21) Appl. No.: 18/725,696

(22) PCT Filed: Jan. 5, 2023
(86) PCT No.: PCT/US2023/010210

§ 371 Date: Jun. 28, 2024

Related U.S. Application Data
(60) Provisional application No. 63/297,066, filed on Jan. 6, 2022.

(51) Int. Cl. A61K 35/28; A61K 31/436; A61K 31/675; A61K 31/7076; A61K 39/00; A61P 37/06; C07K 16/28; C07K 16/42

(52) U.S. Cl. CPC A61K 35/28; A61K 31/436; A61K 31/675; A61K 31/7076; A61P 37/06; C07K 16/2812; C07K 16/2887; C07K 16/4241; A61K 2039/505

(57) ABSTRACT

The present invention provides a clinically applicable method of hematopoietic stem cell transplantation using antibody-based immunosuppression, which can enable engraftment of hematopoietic stem cells. In particular, in Fanconi Anemia this treatment alone is sufficient to enable engraftment of allogeneic hematopoietic stem cells, even in mismatched settings. The methods are optionally combined with CD117 mAb conditioning.

### CROSS REFERENCE TO OTHER APPLICATIONS

This application claims the benefit of U.S. Provisional Application No. 63/297,066, filed Jan. 6, 2022, the contents of which are hereby incorporated by reference in its entirety.

### BACKGROUND

Fanconi Anemia (FA) is a rare inherited disorder caused by dysregulation of genes involved in DNA repair. Currently, 23 genes have been implicated in the FA pathway and mutations in these genes have been shown to lead to defects in DNA repair machinery. Patients suffering from FA may have specific congenital malformations at birth, and moreover have a very high predisposition to bone marrow failure (BMF), myelodysplastic syndrome (MDS), acute myeloid leukemia (AML) and solid tumors due to their genomic instability.

To date, hematopoietic stem cell transplantation (HSCT) is the only curative treatment for the hematological complications of FA which occur in >80-90% of patients. However, although HSCT is a viable option for patients, it has numerous short-term and long-term side effects including graft-versus-host disease (GvHD), infections and organ toxicities. Additionally, FA patients post HSCT have been found to have extremely high rates of additional malignancies, likely due to their underlying DNA repair defect leading to an inherent hypersensitivity to conventional HSCT conditioning agents such as cyclophosphamide, busulfan, and ionizing total body radiation. Due to this, HSCT is often reserved for patients only once they develop transfusion-dependence or have signs of hematologic malignant transformation, despite the benefit of treatment earlier in their disease course.

The use of αCD117 monoclonal antibodies (mAb) as alternative conditioning agents to current genotoxic approaches has been explored. By targeting CD117 (KIT), which is a receptor tyrosine kinase for stem cell factor (SCF) that mediates cell survival, migration, and proliferation in certain cell types, the transient removal of >98% of endogenous HSCs in certain mouse settings and subsequent enhanced donor engraftment post HSCT mice has been shown. See, for example, Czechowicz et al. Science (80). 2007; 318(5854):1296-1299; Chhabra et al. Sci. Transl. Med. 2016; 8(351); Li et al. Nat. Commun. 2019; 10(1):616; Czechowicz et al. Nat. Commun. 2019; 10(1):1-12; Agarwal et al. Biol. Blood Marrow Transplant. 2019; 25(3):S92; Srikanthan et al. Mol. Ther.—Methods Clin. Dev. 2020; 17(June):455-464; Xue X et al. Blood. 2010; 116(24); George, et al. Cell Stem Cell. 2019; 25(2):185-192.e3. (A. Czechowicz et al., 2007; Chhabra et al., 2016; Czechowicz et al., 2019; Li et al., 2019).

Studies with αCD117 mAbs have been initiated in FA mouse models to enable translation into FA patients who have an exceptionally high need for alternative non-genotoxic conditioning. However, prior studies of mAb-based conditioning in FA have not compared the safety and toxicity of these various approaches nor have they extensively explored combination with immunosuppression, which is especially critical to establishing clinically relevant regimens for allogeneic HSCT.

Improved methods for engraftment of hematopoietic stem cells are of great clinical interest. The present invention addresses this need.

### SUMMARY OF THE INVENTION

Compositions and methods are provided for an improved conditioning regimen for engraftment of hematopoietic stem and progenitor cells (HSPC) in an individual, e.g. a human individual. The conditioning regimen is administered prior to the administration of HSPC. In some embodiments the conditioning regimen is free of genotoxic agents. In certain embodiments the conditioning regimen can also be free of HSC-depleting agents. In some embodiments the individual suffers from an anemia, where endogenous HSPC are at a growth disadvantage compared to wild-type HSPC. In some embodiments the individual suffers from Fanconi Anemia (FA). In some embodiments the HSPC are haplo-identical HLA-mismatched relative to the recipient. In some embodiments the HSPC are genetically corrected autologous cells. In some embodiments the HSPC comprise minor mismatches relative to the recipient. In some embodiments the HSPC are expanded or otherwise enhanced compared to wildtype or endogenous HSPC. In some embodiments the HSPC are generated in vitro.

The methods of the disclosure utilize one or a cocktail of immunosuppressive agents as a conditioning regimen for HSPC in a patient, particularly a FA patient, in the absence of genotoxic alkylating agents or radiation. In some embodiments the conditioning is also performed in the absence of HSC-depleting agents, e,g, anti-CD117. In some embodiments the immunosuppressive agent is one or a cocktail of antibodies targeting mature immune cells, e.g. an antibody targeting one or more of CD2, CD3, CD4, CD8, CD40L, CD52, CD122; and anti-thymocyte globulin; etc.

In some embodiments the immunosuppressive agent comprises or consists of an antibody specific for mature T cells, e.g. anti-CD2, anti-CD3, anti-CD4, anti-CD8. In one embodiment a cocktail comprises antibodies specific for CD4, CD8, CD40L, and CD122. In one embodiment a cocktail comprises antibodies specific for CD4 and ATG. In one embodiment the cocktail comprises an antibody that binds to NK cells. In one embodiment a cocktail comprises antibodies specific for CD20, e.g. rituximab; and ATG. In one embodiments a conditioning regimen comprises or consists of: antibodies specific for CD20, e.g. rituximab; ATG; and one or both of cyclophosphamide and fludarabine as immunosuppressive agents.

It is shown herein that immune ablation is sufficient conditioning for successful allogeneic engraftment in FA. In some individuals, genotoxic alkylating HSC-depleting agents and irradiation are not necessary for effective immunoablation. Inclusion of an HSC-targeting agent may result in higher donor chimerism when added to immunoablation chemotherapy, but is not necessary for engraftment, particularly in individuals where there is a competitive growth advantage of wild-type, genetically corrected or otherwise modified HSC. Surprisingly, even in settings lacking HSC depletion in the BM before transplantation, immunosuppressive antibodies, alone or in combination with anti-CD117 antibodies, exhibited high donor engraftment, e.g. at least about 10%, at least about 25%, at least about 40%, at least about 50%, at least about 75% after a period of at least about 10 weeks, at least about 20 weeks, at least about 30 weeks, at least about 40 weeks, at least about 1 year. The level of donor chimerism was observed to increase steadily over time, likely due to a competitive advantage of the transplanted cells to endogenous HSC.

Further, while treatment with immunosuppressive antibodies in the absence of genotoxic agents or HSC-depleting antibodies did not cause significant HSC depletion, it effectively enabled significant donor chimerism in a minor mismatched setting and in haplo-identical HLA-mismatched setting.

In some embodiments, the methods described herein may comprise the steps of: HLA typing a donor and recipient to determine an HLA-matched or HLA-mismatched pair; obtaining hematopoietic cells from the donor comprising CD34⁺ hematopoietic stem and progenitor cells, which may be referred to as HSPC; optionally isolating HSPC of the desired phenotype, e.g. CD34+ cells, and formulating an effective dose of the HSPC; selecting a set of agents for an immunosuppressive conditioning regimen on the recipient prior to infusion of the hematopoietic cells; administering the set of agents for immunosuppressive conditioning; infusing the hematopoietic cells; and monitoring the recipient for hematopoietic stem cell or circulating hematopoietic cell engraftment.

In some embodiments the HSPC are obtained from a donor hematopoietic cell sample. In some embodiments the hematopoietic cell sample is bone marrow. In some embodiments the HSPC are obtained from umbilical cord blood. In some embodiments, the hematopoietic cell sample is obtained by apheresis from donor mobilized peripheral blood. In some embodiments the HSPC are generated in vitro. The HSPC donor may be allogeneic or autologous, for example where the HSPC are genetically engineered by introduction or deletion of genetic material prior to re-infusion, for example during ex vivo culture. Allogeneic donors may be MHC matched to the recipient. The donor may be haploidentical or not haplo-identical to the recipient.

The HSPC are optionally isolated from the hematopoietic cell sample for expression of CD34. HSPC that are purified may be at least about 45% pure, as defined by the percentage of cells that are CD34+ in the population, may be at least about 50% pure, at least about 60% pure, at least about 70% pure, at least about 80% pure, at least about 90% pure. The effective dose of CD34+ cells may be from about 10⁵ to about 10⁷ CD34⁺ cells/kg of recipient body weight, and may be at least about 5×10⁵ CD34⁺ cells/kg of recipient body weight, at least about 10⁶ CD34⁺ cells/kg of recipient body weight, at least about 3×10⁶ CD34+ cells/kg of recipient body weight, at least about 5×10⁶ CD34+ cells/kg of recipient body weight, and may be 10⁷ CD34⁺ cells/kg of recipient body weight or more. The dose of CD34+ cells; the purity of the cells, and the total number of cells delivered, i.e. the total dose of both CD34⁺ and CD34⁻ cells in the infusate, are important parameters for selection of the immunosuppressive conditioning agents.

In some embodiments, the transplantation is performed in the absence of genotoxic agents; and optionally in the absence of HSC-targeting agents. In some embodiments the recipient is an FA patient.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1B. Antagonistic αCD117 mAb treatment inhibits Fancd2^{-/-} Hematopoietic Stem Cell (HSC) growth in vitro with similar effect as on wildtype HSCs. (A) Fancd2^{-/-} HSCs (Lineage⁻Sca-1⁺CD150⁺CD48⁻CD34⁻CD135⁻CD244⁻) growth kinetics via incucyte live-cell imaging system was observed over six days with magnification of 10× after co-culture with various concentrations of αCD117 mAb at 37° C. Fancd2^{-/-} HSC growth was limited in settings of ≥0.1 ug/mL of αCD117 mAb. Cell growth for all treated groups was compared to growth of Fancd2^{-/-} untreated control (Ctl) group. (n=5) (B) Overlay of growth kinetics of C57BL/6J wildtype (WT) and Fancd2^{-/-} (FA) HSCs over six days of co-culture with 0, 0.01 and 100 μg/mL of αCD117 mAb. Cell growth for all treated groups was compared to growth of WT untreated control (Ctl) group. αCD117 mAb inhibition rate of WT and FA HSCs was calculated based on untreated control group of each strain. (n=5) Statistics calculated using unpaired t test with WT control (**** P<0.0001).

FIGS. 2A-2D: Antagonistic αCD117 mAb treatment is insufficient for in vivo Hematopoietic Stem Cell (HSC) depletion in Fancd2^{-/-} mice. (A) Treatment of Fancd2^{-/-} mice with varying doses of αCD117 mAb (500 μg to 2 mg) did not lead to profound depletion of phenotypic long-term (LT) HSCs (Lineage⁻CD117⁺Sca-1⁺CD150⁺CD48⁻) as assessed by flow cytometry or (B) Functional HSC or progenitors by colony forming capacity (CFC) at time of αCD117 mAb clearance. (n=3) (C) Number of LT-HSCs in C57BL/6J wildtype (WT), Rag2^{-/-} and Fancd2^{-/-} bone marrow (BM) and (D) Frequency of LT-HSC was assessed by flow cytometry on day 0, 7, 9, 15, 30 after treatment with 500 ug of αCD117 mAb IV. Only Rag2^{-/-} mice displayed profound decreases in HSC number and frequency after αCD117 mAb treatment (n=5). Statistics calculated using unpaired t test with unconditioned control (**** P<0.0001).

FIGS. 3A-3D: Treatment with αCD117 mAbs leads to host HSC depletion in Fancd2^{-/-} mice and enhanced donor HSC engraftment post syngeneic hematopoietic cell transplantation. (A) Number and frequency of long-term (LT) HSCs (Lineage⁻CD117⁺Sca-1⁺CD150⁺CD48⁻) was assessed in Fancd2^{-/-} FA mice 7 days after treatment with antagonistic αCD117 mAb, αCD117+CD47 mAb, or CD117 antibody-drug conjugates (ADC) compared to untreated controls (n=4-5 per group). The most profound depletion of host HSCs was found in the CD117-ADC group. (B) Peripheral blood donor chimerism (granulocytes, T-cells (CD4⁺ cells and CD8⁺ cells), and B-cells) post near syngeneic transplantation of 20×10⁶ wildtype (WT) CD45.1 whole bone marrow (WBM) cells was observed most profoundly in Fancd2^{-/-} mice conditioned with CD117-ADC. (C) Donor LT-HSC (Lineage⁻CD117⁺Sca-1⁺CD150⁺CD48⁻) chimerism increased gradually at week 4, 24, and 50 post WBM transplantation in all the treatment groups. (D) HSC and progenitor colony forming capacity (CFC) resistance to 50 nM Mitomycin-C (MMC) 50 weeks post-WBM transplantation in 7 days of culture. All the treated and transplanted groups were additionally conditioned with αCD4 mAb to minimize potential immunologic rejection for transplantation due to minor mismatch in genetic background of FA mice with WT donor cells. n=3-4 per group. Statistics calculated using unpaired t test with unconditioned control (**** P<0.0001).

FIGS. 4A-4D: Immunosuppression is required and sufficient to enable donor engraftment in Fancd2^{-/-} mice post syngeneic hematopoietic cell transplantation. (A) No profound phenotypic or functional depletion of HSCs in BM aspiration 7 days after treatment of Fancd2^{-/-} mice with αCD4 mAb and/or αCD117 mAb. n=4 (B) Peripheral blood donor chimerism (granulocytes, T-cells (CD4 and CD8), and B-cells) was similar in Fancd2^{-/-} mice conditioned with αCD4 mAb and αCD117+CD4 mAb at 44 weeks post transplantation of 20×10⁶ wild-type (WT) CD45.1 whole bone marrow (WBM) cells. (C) Donor LT-HSC (Lineage−Sca-1+Kit+CD150+CD48−) BM chimerism was observed in αCD4 mAb conditioning group alone and αCD117+CD4 mAb group at 8, 24, and 44 weeks post syngeneic whole bone marrow (WBM) transplantation with notable (D) HSC and progenitor colony forming capacity (CFC) resistance in these groups to 10 nM Mitomycin-C (MMC) 70 weeks post-WBM transplantation and 7 day culture. n=3-5 per group. Statistics calculated using unpaired t test with unconditioned control (**** P<0.0001).

FIGS. 5A-5C: Immunosuppression is sufficient to enable donor engraftment in Fancd2^{-/-} mice post immunologically mismatched hematopoietic cell transplantation. A) Peripheral blood donor chimerism (granulocytes, T-cells (CD4⁺ cells and CD8⁺ cells), and B-cells) was observed with both immunosuppressant protocol #1 and #2 20 weeks post-transplantation of immunologically mismatched 20×10⁶ WBM cells from the of first generation of C57BL/6J wildtype (WT) crossed with BALB/c. (B) Donor Long-term (LT) HSC (Lineage⁻CD117⁺Sca-1⁺CD150⁺CD48⁻) chimerism increased from 4 to 20 weeks post immunologically mismatched whole bone marrow (WBM) transplantation. (C) HSC and progenitor colony forming capacity (CFC) resistance to 10 nM Mitomycin-C (MMC) was observed 20 weeks post WBM transplantation and 7 day culture. n=3 per group. Statistics calculated using unpaired t test with unconditioned control (**** P<0.0001).

FIGS. 6A-6C: Treatment of Fancd2^{-/-} mice with αCD117 mAbs appears safe with no resulting gross tissue toxicity. (A) Complete blood count analysis of white blood cells (WBC), platelet (PLT), reticulocytes (RET), red blood cells (RBC), hemoglobin (HGB), and hematocrit (HCT) and (B) Analysis of serum levels of blood urea nitrogen (BUN) and creatinine (CREA) to monitor kidney function, and aspartate transaminase (AST) and alanine transaminase (ALT) to monitor liver function in Fancd2^{-/-} mice treated with antagonistic αCD117 mAb, αCD117+CD47 mAb, CD117-ADC, or cyclophosphamide. Blood was taken at day 7 following αCD117 mAb treatments or at day 2 following cyclophosphamide treatment. (C) Representative H&E staining images of thymus and spleen tissue from Fancd2^{-/-} mice treated with various αCD117 mAb or cyclophosphamide which was used as a positive control. Magnification was 40× for thymus and 20× for spleen. (n=5)

FIGS. 7A-7D: Treatment with αCD117 mAbs and αCD4 mAb leads to host HSC and progenitor depletion in Fancd2^{-/-} mice with enhanced donor progenitor engraftment post syngeneic hematopoietic cell transplantation. (A) Profound depletion of BM HSCs was observed in Fancd2^{-/-} mice treated with CD117-ADC compared to untreated controls, but not in (B) BM progenitors (Common Lymphoid Progenitors—CLP, Megakaryocyte-Erythroid Progenitors—MEP, Granulocyte-Monocyte Progenitors—GMP, Common Myeloid Progenitors—CMP, and Myeloid Progenitors—MP). (C) Functional HSC or progenitors as assessed by colony forming capacity (CFC) of BM 7 days after culture showed no difference between animals treated with antagonistic αCD117 mAb, αCD117+CD47 mAb, or CD117-ADC 7 days after mAb treatment. (D) All conditioned animals showed high donor chimerism in all BM progenitor populations assessed 40 weeks after transplantation with 20×10⁶ syngeneic CD45.1 whole bone marrow (WBM) cells. n=4 per group. Statistics calculated using unpaired t test with unconditioned control (**** P<0.0001).

FIGS. 8A-8C: Immunosuppressant protocols used to transplant Fancd2^{-/-} mice with immunologically mismatched hematopoietic cells. (A) Experimental outline for limited immunosuppression with αCD4 mAb and ATG, (B) immunosuppressant conditioning protocol based on George et al. Cell Stem Cell 2019, and (C) immunosuppressant conditioning protocol based on Li et al. Nature Communication 2019. Highlighted yellow texts indicate anti-HSC depletion reagents, which are not included in the immunosuppressant protocol #1 and #2. Red arrow indicates time of whole bone marrow (WBM) transplantation.

FIGS. 9A-9B: Immunosuppressant regimens vary in degree of immune ablation, with only more complex combination regimens resulting in donor engraftment in Fancd2^{-/-} mice post immunologically mismatched hematopoietic cell transplantation. (A) Immune cell composition was determined by peripheral blood flow cytometry prior to immunologically mismatched hematopoietic cell transplantation. T-cells (CD4⁺ cells, CD8⁺ cells, and TCR-Beta cells) were significantly affected with each immunosuppressant regimen. (B) Donor chimerism was established in bone marrow (BM) progenitor sub-populations 20 weeks post transplantation of immunologically mismatched 20×10⁶ WBM cells from the first generation of C57BL/6J wildtype (WT) crossed with BALB/c. n=3 per group. Statistics calculated using unpaired t test with unconditioned control (**** P<0.0001).

FIGS. 10A-10D: Treatment with αCD117 mAbs in combination with immunosuppressants can further enhance donor engraftment in Fancd2^{-/-} mice post immunologically mismatched hematopoietic cell transplantation. (A) Immune cell composition was determined by peripheral blood flow cytometry prior to immunologically mismatched hematopoietic cell transplantation. T-cells (CD4⁺ cells, CD8⁺ cells, and TCR-Beta cells) were significantly affected with each immunosuppressant regimen. (B) Multi-lineage donor peripheral blood chimerism (granulocytes, T-cells (CD4+ cells and CD8+ cells), and B-cells) was observed post George et al. and Li et al. conditioning protocols combined with αCD117 mAb regimens 20 weeks post transplantation of immunologically mismatched 20×10⁶ WBM cells from the of first generation of C57BL/6J wildtype (WT) crossed with BALB/c. (C) Donor long-term (LT) HSC (Lineage⁻CD117⁺Sca-1⁺CD150⁺CD48⁻) chimerism progressively increased from 4 to 20 weeks post immunologically mismatched WBM transplantation with colony forming capacity (CFC) resistance to 10 nM Mitomycin-C (MMC) observed 20 weeks post WBM transplantation and 7 day culture at end of study timepoint. (D) Donor chimerism was also established in BM progenitor sub-populations 20 weeks post immunologically mismatched WBM transplantation. n=3-6 per group. Statistics calculated using unpaired t test with unconditioned control (**** P<0.0001).

FIG. 11. Table showing similar clearance of antagonistic αCD117 mAb in Fancd2^{-/-} mice regardless of treatment dose. Fancd2^{-/-} mice, age range from 8 weeks old to 12 weeks old, were treated with varying doses of αCD117 mAb via either via intraperitoneal (IP) or intravenous (IV) injection. Peripheral blood serum was analyzed by flow cytometry every other day from day 1 to 9 after αCD117 mAb treatment. All doses of αCD117 mAb resulted to similar mAb persistence in circulation with detectable mAb clearance from the peripheral blood by 7 days after treatment in all animals. n=3 per group.

FIG. 12. Degree of immune suppression required varies with degree of immunologic mismatch between donor and host, with varying donor granulocyte chimerism after WBM transplantation of Fancd2^{-/-} mice depending upon the donor WBM and the conditioning regimen as indicated. High donor granulocyte chimerism was observed up to 16 weeks post transplantation of minor mismatched wild-type CD45.1 pepboy (B6.SJL-Ptprc^{a}Pepc^{b}/Boy) WBM into Fancd2^{-/-} (C57BL/6N—129) mice with anti-CD4 mAb (GK1.5) conditioning and without the need for HSC-ablation. In fully immunologically matched settings upon transplantation of wild-type CD45.1^{pm} (C57BL/6N) WBM into Fancd2^{-/-} (C57BL/6N—129) mice, high donor granulocyte chimerism up to 16 weeks post transplantation was observed in unconditioned settings without the need for immune suppression or HSC-ablation. No donor engraftment was observed in non-transplanted Fancd2^{-/-} (C57BL/6N—129) mice or upon transplantation of minor mismatched wild-type CD45.1 pepboy (B6.SJL-Ptprc^{a}Pepc⁰/Boy) WBM into unconditioned Fancd2^{-/-} (C57BL/6N—129) mice.

### DETAILED DESCRIPTION OF THE EMBODIMENTS

It is an objective of the present invention to provide a new clinically applicable method of stem cell transplantation which facilitates engraftment and reconstitutes immunocompetence of the recipient without requiring radiotherapy or chemotherapy, or development of GVHD or graft rejection.

To facilitate an understanding of the invention, a number of terms are defined below.

Before the present active agents and methods are described, it is to be understood that this invention is not limited to the particular methodology, products, apparatus and factors described, as such methods, apparatus and formulations may, of course, vary. It is also to be understood that the terminology used herein is for the purpose of describing particular embodiments only, and is not intended to limit the scope of the present invention which will be limited only by appended claims.

It must be noted that as used herein and in the appended claims, the singular forms “a,” “and,” and “the” include plural referents unless the context clearly dictates otherwise. Thus, for example, reference to “a drug candidate” refers to one or mixtures of such candidates, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Generally, conventional methods of protein synthesis, recombinant cell culture and protein isolation, and recombinant DNA techniques within the skill of the art are employed in the present invention. Such techniques are explained fully in the literature, see, e.g., Maniatis, Fritsch & Sambrook, Molecular Cloning: A Laboratory Manual (1982); Sambrook, Russell and Sambrook, Molecular Cloning: A Laboratory Manual (2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

### Definitions

Conditioning regimen. Patients undergoing an allogeneic hemopoietic stem cell transplant (HSCT), are conventionally prepared with a “conditioning regimen” that may suppress the recipient's immune system and/or deplete endogenous stem cells, in order to allow engraftment of the donor stem cells.

The intensity of conventional conditioning regimens can vary significantly. Description of the regimens can refer to genotoxic or immunosuppressive regimens, which may overlap with reference to myeloablative or non-myeloablative regimens. See, for example, Bacigalupo et al. (2009) Biol Blood Marrow Transplant. 15(12):1628-1633, herein specifically incorporated by reference.

Genotoxic regimens comprise, at least in part, the administration of agents with direct or indirect effects on the DNA: the induction of mutations, mistimed event activation, and direct DNA damage leading to mutations. Examples of genotoxic agents include radiation and certain chemotherapeutic drugs, such as alkylating agents, intercalating agents and inhibitors of enzymes involved in DNA replication. In some embodiments, the methods of the invention are immunosuppressive, and exclude the use of such agents. In some embodiments, the methods of the invention are immunosuppressive, and exclude the use of alkylating agents and radiation, but may include agents such as cyclophosphamide and fludarabine.

The conditioning regimens provided herein are immunosuppressive and non-myeloablative, and utilize targeted agents for depletion of endogenous cells that prevent engraftment, without causing long-lasting pancytopenia.

Immunosuppressive agent. An immunosuppressive agent blocks the activity of immune cells, particularly T lymphocytes, for a short period of time, usually the period of time at or shortly before the administration of the donor cells. Transient immunosuppression, i.e. an effective serum level of the immunosuppressive agent(s) may be maintained for at least about 1 day, at least about 3 days, at least about 1 week, at least about 2 weeks, at least about 3 weeks, at least about 4 weeks, at least about 5 weeks, at least about 6 weeks, and may be maintained for up to 1 month, up to 2 months, up to 3 months, up to 4 months, up to 5 months, up to 6 months, or more. In some embodiments a single dose of the agent is administered immediately prior to, or concomitantly, with the donor cells. The initial dose of the agent may be made within about 2 weeks, within about 1 week, within about 3 days, within about 2 days, within about 1 day, or at the time of administration of the donor cells.

Transient immunosuppression can be achieved by administration of a pharmacologic immunosuppressive agent, including without limitation calcineurin inhibitors, which combine with binding proteins to inhibit calcineurin activity, and which include, for example, tacrolimus, cyclosporine A, etc. Levels of both cyclosporine and tacrolimus must be carefully monitored. Initially, levels can be kept in the range of 10-20 ng/mL, but, after 3 months, levels may be kept lower (5-10 ng/mL) to reduce the risk of nephrotoxicity. Other pharmacologic agents for this purpose include steroids, azathioprine, mycophenolate mofetil, rapamycin, sirolimus, etc. Immunosuppressants may also include kinase inhibitors, such as Ruxolitinib; and JAK inhibitors.

In some embodiments a transient immunosuppressive agent blocks the interaction of CD40 and CD40 ligand. CD40 is a costimulatory protein found on antigen presenting cells (APCs) and is required for their activation. These APCs include phagocytes (macrophages and dendritic cells) and B cells. CD40 is part of the TNF receptor family. The primary activating signaling molecules for CD40 are IFNγ and CD40 ligand (CD40L).

T cell and B cell ablation. It is desirable to delete endogenous T cells, optionally in combination with depletion of B cells. In some embodiments an immunosuppressive agent is specific for T cells, in others it also acts on NK cells. Antibodies that target T cells include, for example, antibodies specific for CD2, CD3, CD4, CD8, CD52 (campath), CD45, and ATG.

Anti-thymocyte globulin (ATG) is a polyclonal IgG produced by immunizing rabbits, horses, or goats with human lymphocytes and subsequently purifying the antibody from sera. Rabbit ATG (rATG) is now the predominant form of ATG used clinically. Several forms of rATG are in existence depending upon manufacturer and regulatory agency, with Thymoglobulin® being the most commonly used agent in the United States and Grafalon® being the most commonly used agent in Europe. ATG targets a wide variety of cell surface receptors on T-cells, B-cells and NK-cells, including CD2, CD3, CD4, CD8, CD11a, CD18, CD25 and CD45. Upon administration, it is able to induce rapid and profound T-cell depletion.

CD3 is a protein complex associated with the T-cell receptor (TCR) that is crucial for signal transduction upon TCR recognition of antigen. Since inhibition of CD3 blocks T-cell activation, antibodies such as OKT3 (Janssen-Cilag), also known as muromonab, is used for this purpose. Multiple anti-human CD3 and CD2 mAb are in clinical development, including Teplizumab, and MGA031, is a humanized IgG1 antibody that was developed by grafting the complementarity determining region of OKT3 into a human IgG1 backbone. Otelixizumab (ChAglyCD3, TRX4, GSK2136525) is derived from the rat antibody YTH12.5, and is a humanized IgG1 with a single mutation in the γ1 Fc portion to avoid glycosylation and thus inhibit FcR binding. Visilizumab (Nuvion, HuM291) is a humanized IgG2 antibody rendered non mitogenic by two point mutations in its Fc region. Foralumab (28F11-AE; NI-0401) is an entirely human anti-CD3 mAb. BTI-322 and Siplizumab are human anti-CD2 mAb in development.

A useful agent for depletion of T cells and NK cells is an anti-CD52 antibody, exemplified by the clinically approved antibody Campath (alemtuzumab), which is a recombinant DNA-derived humanized monoclonal antibody directed against the 21-28 kD cell surface glycoprotein, CD52. Campath-1H is an IgG1 kappa antibody with human variable framework and constant regions, and complementarity-determining regions from a murine (rat) monoclonal antibody (Campath-1G). Campath may be administered, for example, at the currently accepted clinical dose, e.g. escalating to the maximum single dose of 30 mg over a period of from about 3 to about 7 days. As CD52 is expressed on 95% of peripheral blood lymphocytes, monocytes and thymocytes, Alemtuzumab is able to dramatically deplete over 99% of lymphocytes with a single injection. Alemtuzumab is thought to primarily induce cell death upon binding to CD52 through complement-mediated cytolysis, antibody-mediated cytotoxicity and apoptosis.

Although the allograft immune response appears to be mainly driven by T-cells, B-cells also contribute. In addition to differentiating into antibody-secreting plasma cells, B-cells shape the T-cell response through a combination of antigen presentation, cytokine production and co-stimulation. Furthermore, B-cells have direct effects on the allograft that can be initiated by ischemic injury. Rituximab, an anti-CD20 monoclonal antibody, has been used prominently for this purpose.

The interaction between CD40 and CD40L is another T-cell co-stimulatory pathway. CD40 is also expressed on B cells, various antigen-presenting cells (APCs), including dendritic cells (DCs), monocytes, platelets, and macrophages as well as by non-hematopoietic cells such as myofibroblasts, fibroblasts, epithelial, and endothelial cells. CD40 ligand (CD40L or CD154) is primarily found on activated T-cells, activated B-cells and platelets; under inflammatory conditions it is also induced on NK-cells, monocytic cells, mast cells, and basophils. Several anti-CD40L antibodies have been developed for use in transplantation, such as ruplizumab (Biogen), toralizumab (IDEC Pharmaceuticals), ABI793 (Novartis), H106 and BMS-986004 (Bristol Myers-Squibb). Ruplizumab, or hu5c8, is a humanized IgG1 antibody. Toralizumab, known as IDEC-31, is also a humanized IgG1. ABI793 is fully humanized. As an alternative to anti-CD40L antibodies, chimeric antibodies anti-CD40 antibodies ch5D12 and chi220 are also immunosuppressive. 4D11 is a human anti-CD40 antibody. CFZ533 is a fully human, Fc-silenced, non-depleting, IgG1 mAb that blocks CD40 pathway signaling and activation of CD40⁺ cell types.

CD2, or LFA-2, is an adhesion molecule and a member of the Ig superfamily expressed on T-cells, thymocytes, and NK-cells. Targeting CD2 to induce tolerance was first accomplished through the fusion protein alefacept (Astellas Pharma US), formed from CD58 and the constant portion of human IgG1. A humanized anti-CD2 antibody derived from BTI-322, siplizumab (MEDI-507) has a unique three-in-one mode of action: selective T-cell depletion with relative sparing and upregulation of regulatory T cells (Tregs).

With respect to timing, a T cell and/or B cell depleting agent is desirably active in the period of time at or shortly before the administration of the donor cells. Therapeutic levels of the depletion agent may be maintained for at least about 3 days, at least about 1 week, at least about 2 weeks, at least about 3 weeks, at least about 4 weeks, at least about 5 weeks, at least about 6 weeks, and may be maintained for up to 1 month, up to 2 months, up to 3 months, up to 4 months, up to 5 months, up to 6 months, or more following administration of the donor cells. In some embodiments a dose of the agent is administered within about 3 days, within about 2 days, within about 1 day, or at the time of administration of the donor cells, and depending on the antibody, may be administered daily for several days, e.g. 2, 3 4 etc., prior to infusion. An effective dose of an antibody may be up to about 50 mg/kg, up to about 25 mg/kg; up to about 10 mg/kg, up to about 5 mg/kg; up to about 1 mg/kg; up to about 0.5 mg/kg; or less, for example up to about 100 g/kg, up to about 50 g/kg, up to about 10 g/kg, up to about 1 g/kg, where the dose may vary with the specific antibody and recipient.

Antibody-based therapy may use monoclonal (e.g., muromonab-CD3) or polyclonal antibodies; anti-CD25 antibodies (e.g., basiliximab, daclizumab), etc. Antibodies include, for example, an ATG preparation, a-OKT3, BTI-322® (U.S. Pat. No. 5,730,979 the disclosure of which is hereby incorporated by reference), and Rituximab.

NK cell ablation. It may also be desirable to deplete endogenous NK cells. As indicated above, some agents act on both T cells and NK cells, e.g. antibodies to CD2, CD52, etc. Other agents are specific for NK cells and may be administered in combination with T cell targeted agents. Antibodies that selectively target NK cells include, for example, antibodies specific for CD122 and CD56.

With respect to timing, an NK cell depleting agent is desirably active in the period of time at or shortly before the administration of the donor cells. Therapeutic levels of the depletion agent may be maintained for at least about 1 day, at least about 3 days, at least about 1 week, at least about 2 weeks, at least about 3 weeks, at least about 4 weeks, at least about 5 weeks, at least about 6 weeks, and may be maintained for up to 1 month, up to 2 months, up to 3 months, up to 4 months, up to 5 months, up to 6 months, or more following administration of the donor cells. In some embodiments a dose of the agent is administered within about 3 days, within about 2 days, within about 1 day, or at the time of administration of the donor cells, and depending on the antibody, may be administered daily for several days, e.g. 2, 3, 4 etc., prior to infusion. An effective dose of an antibody may be up to about 50 mg/kg, up to about 25 mg/kg; up to about 10 mg/kg, up to about 5 mg/kg; up to about 1 mg/kg; up to about 0.5 mg/kg; or less, for example up to about 100 μg/kg, up to about 50 μg/kg, up to about 10 μg/kg, up to about 1 μg/kg, where the dose may vary with the specific antibody and recipient.

“CD122” (also called “interleukin-2 receptor subunit beta”, IL2RB) is a type I membrane protein. CD122 is a subunit of the interleukin 2 receptor (IL2R), which is involved in T cell-mediated immune responses, and is present in 3 forms with respect to ability to bind interleukin 2. The low affinity form of IL2R is a monomer of the alpha subunit and is not involved in signal transduction. The intermediate affinity form consists of an alpha/beta subunit heterodimer, while the high affinity form consists of an alpha/beta/gamma subunit heterotrimer. Both the intermediate and high affinity forms of the receptor are involved in receptor-mediated endocytosis and transduction of mitogenic signals from interleukin 2. The use of alternative promoters results in multiple transcript variants encoding the same protein.

As used herein, the term “anti-CD122 agent” or “agent that provides for CD122 blockade” refers to any agent that depletes CD122 positive cells, including natural killer (NK) cells. Non-limiting examples of suitable anti-CD122 reagents include anti-IL-2 antibodies, and anti-CD122 antibodies or antibody fragments.

Antibodies that target CD56 are in clinical development and find use in NK cell depletion. For example, IMGN901 is a CD56-targeting antibody-drug conjugate designed for selective delivery of the cytotoxic maytansinoid DM1 with a maximum tolerated dose (MTD) of about 75 mg/m². and which may be administered at doses of, for example, from about 1 to about 60 mg/m².

“Concomitant administration” of active agents in the methods of the invention means administration with the reagents at such time that the agents will have a therapeutic effect at the same time. Such concomitant administration may involve concurrent (i.e. at the same time), prior, or subsequent administration of the agents. A person of ordinary skill in the art would have no difficulty determining the appropriate timing, sequence and dosages of administration for particular drugs and compositions of the present invention.

“Major histocompatibility complex antigens” (“MHC”, also called “human leukocyte antigens”, HLA) are protein molecules expressed on the surface of cells that confer a unique antigenic identity to these cells. MHC/HLA antigens are target molecules that are recognized by T-cells and natural killer (NK) cells as being derived from the same source of hematopoietic stem cells as the immune effector cells (“self”) or as being derived from another source of hematopoietic reconstituting cells (“non-self”). Two main classes of HLA antigens are recognized: HLA class I and HLA class II. HLA class I antigens (A, B, and C in humans) render each cell recognizable as “self,” whereas HLA class II antigens (DR, DP, and DQ in humans) are involved in reactions between lymphocytes and antigen presenting cells. Both have been implicated in the rejection of transplanted organs.

An important aspect of the HLA gene system is its polymorphism. Each gene, MHC class I (A, B and C) and MHC class II (DP, DQ and DR) exists in different alleles. HLA alleles are designated by numbers and subscripts. For example, two unrelated individuals may carry class I HLA-B, genes B5, and Bw41, respectively. Allelic gene products differ in one or more amino acids in the α and/or β domain(s). Large panels of specific antibodies or nucleic acid reagents are used to type HLA haplotypes of individuals, using leukocytes that express class I and class II molecules. The genes most important for HLA typing are the six MHC Class I and Class II proteins, two alleles for each of HLA-A; HLA-B and HLA-DR.

The HLA genes are clustered in a “super-locus” present on chromosome position 6p21, which encodes the six classical transplantation HLA genes and at least 132 protein coding genes that have important roles in the regulation of the immune system as well as some other fundamental molecular and cellular processes. The complete locus measures roughly 3.6 Mb, with at least 224 gene loci. One effect of this clustering is that “haplotypes”, i.e. the set of alleles present on a single chromosome, which is inherited from one parent, tend to be inherited as a group. The set of alleles inherited from each parent forms a haplotype, in which some alleles tend to be associated together. Identifying a patient's haplotypes can help predict the probability of finding matching donors and assist in developing a search strategy, because some alleles and haplotypes are more common than others and they are distributed at different frequencies in different racial and ethnic groups.

As used herein, the term “HLA matched” refers to a donor recipient pair in which none of the HLA antigens are mismatched between the donor and recipient. HLA matched (i.e., where all of the 6 alleles are matched) donor/recipient pairs have a decreased risk of graft v. host disease (GVHD) relative to mismatched pairs (i.e. where at least one of the 6 alleles is mismatched). HLA haploidentical refers to a match where one chromosome is matched at least at HLA-A; HLA-B and HLA-DR, and may be matched at minor histocompatibility loci on the chromosome; but is not necessarily matched on the second chromosome. Such donors frequently occur in families, e.g. a parent is haploidentical to a child; and siblings may be haploidentical.

As used herein, the term “HLA mismatched” refers to a donor recipient pair in which at least one HLA antigen, in particular with respect to HLA-A, HLA-B and HLA-DR, is mismatched between the donor and recipient. In some cases, one haplotype is matched and the other is mismatched. This situation is frequently found with organs from living or deceased donors. HLA mismatched donor/recipient pairs have an increased risk of GVHD relative to perfectly matched pairs (i.e. where all 6 alleles are matched).

HLA alleles are typically noted with a variety of levels of detail. Most designations begin with HLA- and the locus name, then * and some (even) number of digits specifying the allele. The first two digits specify a group of alleles. Older typing methodologies often could not completely distinguish alleles and so stopped at this level. The third through fourth digits specify a synonymous allele. Digits five through six denote any synonymous mutations within the coding frame of the gene. The seventh and eighth digits distinguish mutations outside the coding region. Letters such as L, N, Q, or S may follow an allele's designation to specify an expression level or other non-genomic data known about it. Thus, a completely described allele may be up to 9 digits long, not including the HLA-prefix and locus notation.

As used herein, a “recipient” is an individual to whom an organ, tissue or cells from another individual (donor), commonly of the same species, has been transferred. For the purposes of the present disclosure, a recipient and a donor are either HLA-matched or HLA-mismatched.

C-kit (CD117). CD117 is a receptor tyrosine kinase type Ill, which binds to stem cell factor (a substance that causes certain types of cells to grow), also known as “steel factor” or “c-kit ligand”. When this receptor binds to stem cell factor (SCF) it forms a dimer that activates its intrinsic tyrosine kinase activity, that in turn phosphorylates and activates signal transduction molecules that propagate the signal in the cell. See, for example, the human refseq entries Genbank NM_000222; NP_000213. CD117 is an important cell surface marker used to identify certain types of hematopoietic (blood) progenitors in the bone marrow. Hematopoietic stem cells (HSC), multipotent progenitors (MPP), and common myeloid progenitors (CMP) express high levels of CD117. A number of antibodies that specifically bind CD117 are known in the art, including without limitation 2B8, ACK2, YB5-B8, 57A5, 104D2, SR1, AMG191/JSP191 etc. The optional use of these antibodies targets HSC for depletion.

As used herein, “antibody” includes reference to an immunoglobulin molecule immunologically reactive with a particular antigen, and includes both polyclonal and monoclonal antibodies. Antibodies may be “entire” antibodies comprising an Fc region, e.g. a human IgG1, IgG2a, IgG2b, IgG3, IgG4, etc. Fc region sequence. The term also includes genetically engineered forms such as chimeric antibodies (e.g., humanized murine antibodies) and heteroconjugate antibodies. The term “antibody” also includes antigen binding forms of antibodies, including fragments with antigen-binding capability (e.g., Fab′, F(ab′)₂, Fab, Fv and rIgG. The term also refers to recombinant single chain Fv fragments (scFv). The term antibody also includes bivalent or bispecific molecules, diabodies, triabodies, and tetrabodies.

Selection of antibodies for endogenous stem cell ablation may be based on a variety of criteria, including selectivity, affinity, cytotoxicity, etc. The phrase “specifically (or selectively) binds” to an antibody or “specifically (or selectively) immunoreactive with,” when referring to a protein or peptide, refers to a binding reaction that is determinative of the presence of the protein, in a heterogeneous population of proteins and other biologics. Thus, under designated immunoassay conditions, the specified antibodies bind to a particular protein sequences at least two times the background and more typically more than 10 to 100 times background. In general, antibodies of the present invention bind antigens on the surface of target cells in the presence of effector cells (such as natural killer cells or macrophages). Fc receptors on effector cells recognize bound antibodies. The cross-linking of Fc receptors signals the effector cells to kill the target cells by cytolysis or apoptosis. In one embodiment, the induction is achieved via antibody-dependent cellular cytotoxicity (ADCC). In alternative embodiments, the antibodies are active in growth inhibition of the targeted cells, an ablation is achieved by interfering with growth factor signaling, e.g. antibodies specific for growth factor receptors such as c-kit.

An antibody immunologically reactive with a particular antigen can be generated by recombinant methods such as selection of libraries of recombinant antibodies in phage or similar vectors, or by immunizing an animal with the antigen or with DNA encoding the antigen. Methods of preparing polyclonal antibodies are known to the skilled artisan. The antibodies may, alternatively, be monoclonal antibodies. Monoclonal antibodies may be prepared using hybridoma methods. In a hybridoma method, an appropriate host animal is typically immunized with an immunizing agent to elicit lymphocytes that produce or are capable of producing antibodies that will specifically bind to the immunizing agent. Alternatively, the lymphocytes may be immunized in vitro. The lymphocytes are then fused with an immortalized cell line using a suitable fusing agent, such as polyethylene glycol, to form a hybridoma cell.

Human antibodies can be produced using various techniques known in the art, including phage display libraries. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire.

Antibodies also exist as a number of well-characterized fragments produced by digestion with various peptidases. Thus pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into a Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin.

Antibodies of interest for ablation may be tested for their ability to induce ADCC (antibody-dependent cellular cytotoxicity). Antibody-associated ADCC activity can be monitored and quantified through detection of either the release of label or lactate dehydrogenase from the lysed cells, or detection of reduced target cell viability (e.g. annexin assay). Assays for apoptosis may be performed by terminal deoxynucleotidyl transferase-mediated digoxigenin-11-dUTP nick end labeling (TUNEL) assay (Lazebnik et al., Nature: 371, 346 (1994). Cytotoxicity may also be detected directly by detection kits known in the art, such as Cytotoxicity Detection Kit from Roche Applied Science (Indianapolis, Ind.). Preferably, the antibodies of the present invention induce at least 10%, 20%, 30%, 40%, 50%, 60%, or 80% cytotoxicity of the target cells.

In some embodiments, the antibody is conjugated to an effector moiety. The effector moiety can be any number of molecules, including labeling moieties such as radioactive labels or fluorescent labels, or can be a cytotoxic moiety, which may be referred to as an antibody drug conjugate (ADC). Cytotoxic agents are numerous and varied and include, but are not limited to, cytotoxic drugs or toxins or active fragments of such toxins. Suitable toxins and their corresponding fragments include amatoxin, amanitin, calicheamicin, pyrrolobenzodiazepine, saporin, diphtheria A chain, exotoxin A chain, ricin A chain, abrin A chain, curcin, crotin, phenomycin, enomycin, auristatin-E and the like. Cytotoxic agents also include radiochemicals made by conjugating radioisotopes to antibodies. Targeting the cytotoxic moiety to transmembrane proteins serves to increase the local concentration of the cytotoxic moiety in the targeted area.

Stem cells of interest for transplantation are hematopoietic stem cells. The cells of interest are typically mammalian, where the term refers to any animal classified as a mammal, including humans, domestic and farm animals, and zoo, laboratory, sports, or pet animals, such as dogs, horses, cats, cows, mice, rats, rabbits, etc. Preferably, the mammal is human.

For engraftment purposes, a composition comprising hematopoietic stem cells is administered to a patient. Such methods are well known in the art. The stem cells are optionally, although not necessarily, enriched or purified. Abundant reports explore various methods for enrichment or purification of stem cells and subsequent engraftment, including flow cytometry; an isolex system (Klein et al. (2001) Bone Marrow Transplant. 28(11):1023-9; Prince et al. (2002) Cytotherapy 4(2):137-45); immunomagnetic separation (Prince et al. (2002) Cytotherapy 4(2):147-55; Handgretinger et al. (2002) Bone Marrow Transplant. 29(9):731-6; Chou et al. (2005) Breast Cancer. 12(3):178-88); and the like. Each of these references is herein specifically incorporated by reference, particularly with respect to procedures, cell compositions and doses for hematopoietic stem cell transplantation.

Hematopoietic stem cells can be obtained by harvesting from bone marrow or from peripheral blood. Bone marrow is generally aspirated from the posterior iliac crests while the donor is under either regional or general anesthesia. Additional bone marrow can be obtained from the anterior iliac crest. A dose of 1×10⁸ and 2×10⁸ marrow mononuclear cells per kilogram is generally considered desirable to establish engraftment in autologous and allogeneic marrow transplants, respectively. Bone marrow can be primed with granulocyte colony-stimulating factor (G-CSF; filgrastim [Neupogen]) to increase the stem cell count.

Mobilization of stem cells from the bone marrow into peripheral blood by cytokines such as G-CSF or GM-CSF has led to the widespread adoption of peripheral blood progenitor cell collection by apheresis for hematopoietic stem cell transplantation. The dose of G-CSF used for mobilization is 10 g/kg/day. In autologous donors who are heavily pretreated, however, doses of up to 40 g/kg/day can be given. Mozobil may be used in conjunction with G-CSF to mobilize hematopoietic stem cells to peripheral blood for collection.

Current guidelines indicate that the minimum dose required for engraftment is 1-2×10⁶ CD34⁺ cells/kg body weight for autologous and allogeneic transplants. Higher doses would result in better engraftment, but doses in the range of 8×10⁶ may be associated with increased risk of extensive GVHD.

The cells which are employed may be fresh, frozen, or have been subject to prior culture. They may be fetal, neonate, adult, etc. Hematopoietic stem cells may be obtained from fetal liver, bone marrow, blood, particularly G-CSF or GM-CSF mobilized peripheral blood, or any other conventional source. Cells for engraftment are optionally isolated or derived from other cells, where the manner in which the stem cells are separated from other cells of the hematopoietic or other lineage is not critical to this invention. If desired, a substantially homogeneous population of stem or progenitor cells may be obtained by selective isolation of cells free of markers associated with differentiated cells, while displaying epitopic characteristics associated with the stem cells.

Cells may be genetically altered in order to introduce genes useful in the differentiated cell, e.g. repair of a genetic defect in an individual, selectable marker, etc., usually to correct a genetic defect causing disease. A vector is designed using the known encoding sequence for the desired gene, operatively linked to a promoter that is constitutive, pan-specific, specifically active in a differentiated cell type, etc. Suitable inducible promoters are activated in a desired target cell type, either the transfected cell, or progeny thereof. By transcriptional activation, it is intended that transcription will be increased above basal levels in the target cell by at least about 100 fold, more usually by at least about 1000 fold. Various promoters are known that are induced in different cell types.

Gene editing technologies such as CRISPR/CAS9 systems can be used for altering genes in the transplanted cells. Alternatively, many vectors useful for transferring exogenous genes into target mammalian cells are available. The vectors may be episomal, e.g. plasmids, virus derived vectors such cytomegalovirus, adenovirus, etc., or may be integrated into the target cell genome, through homologous recombination or random integration, e.g. retrovirus derived vectors such MMLV, HIV-1, ALV, etc. For modification of stem cells, lentiviral vectors are preferred. Lentiviral vectors such as those based on HIV or FIV gag sequences can be used to transfect non-dividing cells, such as the resting phase of human stem cells. Combinations of retroviruses and an appropriate packaging line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line. The vectors may include genes that must later be removed, e.g. using a recombinase system such as Cre/Lox, or the cells that express them destroyed, e.g. by including genes that allow selective toxicity such as herpesvirus TK, bcl-xs, etc.

Chimerism, as used herein, generally refers to chimerism of the hematopoietic system, unless otherwise noted. A determination of whether an individual is a full chimera, mixed chimera, or non-chimeric made be made by an analysis of a hematopoietic cell sample from the graft recipient, e.g. peripheral blood, bone marrow, etc. as known in the art. Analysis may be done by any convenient method of typing. In some embodiments the degree of chimerism amongst all mononuclear cells, T cells, B cells, CD56+ NK cells, and CD15+ neutrophils is regularly monitored, using PCR with probes for microsatellite analysis. For example, commercial kits that distinguish polymorphisms in short terminal repeat lengths of donor and host origin are available. Automated readers provide the percentage of donor type cells based on standard curves from artificial donor and host cell mixtures.

For autologous gene-modified cells, the level of gene correction in the targeted cell population may be monitored, which provides an equivalent of chimerism.

Individuals who exhibited more than a 95% donor cells in a given blood cell lineage by such analysis at any time post-transplantation are referred to as having full donor chimerism in this transplant patient group. Mixed chimerism is defined as greater than 1% donor but less than 95% donor DNA in such analysis. Individuals who exhibit mixed chimerism may be further classified according to the evolution of chimerism, where improving mixed chimerism is defined as a continuous increase in the proportion of donor cells over at least a 6-month period. Stable mixed chimerism is defined as fluctuations in the percentage of recipient cells over time, without complete loss of donor cells.

A “patient” for the purposes of the present invention includes both humans and other animals, particularly mammals, including pet and laboratory animals, e.g. mice, rats, rabbits, etc. Thus, the methods are applicable to both human therapy and veterinary applications. In one embodiment the patient is a mammal, preferably a primate. In other embodiments the patient is human.

Additional terms. The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with cancer, those with an infection, etc.) as well as those in which prevention is desired (e.g., those with increased susceptibility to cancer, those with an increased likelihood of infection, those suspected of having cancer, those suspected of harboring an infection, etc.).

### Methods for Engraftment

The methods of the invention provide for engraftment of stem cells after transplantation into a recipient. The recipient may be immunocompetent, and the transplantation may be performed in the absence of genotoxic conditioning, i.e. in the absence of radiation and/or chemotherapeutic drugs. The recipient is conditioned with one or a cocktail of immunosuppressive antibodies, optionally in the absence of HSC-targeting antibodies. Following the conditioning regimen, an effective dose of a cellular composition comprising exogenous stem cells is administered to the recipient. The stem cells may be autologous, allogeneic or xenogeneic.

The conditioning agents, which may be provided in the absence of myeloablative radiation or chemotherapy, are administered daily, twice daily, every other day, every third day, etc. for a period of time sufficient to achieve the desired immunosuppression, at least about 1 day, up to about 2 days, up to about 3, 4, 5, 6, 7, 8, 9 or more days. In some embodiments from 4-9 days is sufficient. The agents may be formulated together or separately, but are administered concomitantly. “Concomitant” and “concomitantly” as used herein refer to the administration of at least two agents, or at least three agents, to a patient either simultaneously or within a time period during which the effects of the first administered agent are still operative in the patient.

In methods of use, each antibody in a cocktail is administered in a dose effective to achieve immunosuppression of the targeted cells. The effective dose may be from about 1 ng/kg weight, 10 ng/kg weight, 100 ng/kg weight, 1 g/kg weight, 10 g/kg weight, 25 g/kg weight, 50 g/kg weight, 100 g/kg weight, 250 g/kg weight, 500 g/kg weight, 750 μg/kg weight, 1 mg/kg weight, 5 mg/kg weight, 10 mg/kg weight, 25 mg/kg weight, 50 mg/kg weight, 75 mg/kg weight, 100 mg/kg weight, 250 mg/kg weight, 500 mg/kg weight, 750 mg/kg weight, for example up to about 500 mg/kg weight, and the like. The dosage may be administered multiple times as needed, e.g. every 12 hours, every 18 hours, daily, every 2 days, every 3 days, weekly, and the like. The compositions can be administered in a single dose, or in multiple doses, usually multiple doses over a period of time. Determining an effective amount of an agent according to the present methods can be done based on animal data using routine computational methods.

Typically antibodies are purified and/or isolated. Specifically, as used herein, an “isolated” or “purified” polypeptide or protein is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. Purified also defines a degree of sterility

Compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, the subject compound is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures. In some embodiments, the subject compound is formulated for sustained release.

Each of the active immunosuppressive can be provided in a unit dose of from about 0.1 μg, 0.5 μg, 1 μg, 5 μg, 10 μg, 50 μg, 100 μg, 500 μg, 1 mg, 5 mg, 10 mg, 50, mg, 100 mg, 250 mg, 500 mg, 750 mg or more.

The infusion of HSPCs is a relatively simple process that is performed at the bedside. The bone marrow product is generally used fresh and is infused through a central vein over a period of several hours. Autologous products are frequently cryopreserved; if so, they are thawed at the bedside and infused rapidly over a period of several minutes. The dose of HSC is at least about 10⁵ CD34⁺ cells/kg body weight, at least about 0.5×10⁶, at least about 10⁶, and up to about 2.5×10⁶, 5×10⁶, 7.5×10⁶, 10⁷ CD34⁺ cells/kg body weight.

Where the donor is allogeneic to the recipient, the HLA type of the donor and recipient may be tested for a match. Traditionally, the loci critical for matching are HLA-A, HLA-B, and HLA-DR. HLA-C and HLA-DQ are also now considered when determining the appropriateness of a donor. A completely matched sibling donor is generally considered the ideal donor. For unrelated donors, a complete match or a single mismatch is considered acceptable for most transplantation, although in certain circumstances, a greater mismatch is tolerated. Preferably matching is both serologic and molecular. Where the donor is umbilical cord blood the degree of tolerable HLA disparity is much greater, and a match of 3-4 out of the 6 HLA-A, HLA-B and HLA-DRB1 antigens is sufficient for transplantation. Immunocompetent donor T cells may be removed using a variety of methods to reduce or eliminate the possibility that graft versus host disease (GVHD) will develop.

For positive selection of CD34⁺ cells, commercial instruments can be employed to remove the desired cells, using solid-phase, anti-CD34 monoclonal antibodies. With negative selection, anti-cancer or anti-immune cell monoclonal antibodies can be used to remove tumor cells or immune cells respectively, leaving stem cells in the graft.

For immunosuppression, the antibodies are formulated in a pharmaceutical composition. The exact dose will depend on the purpose of the treatment, and will be ascertainable by one skilled in the art using known techniques (e.g., Ansel et al., Pharmaceutical Dosage Forms and Drug Delivery; Lieberman, Pharmaceutical Dosage Forms (vols. 1-3, 1992), Dekker, ISBN 0824770846, 082476918X, 0824712692, 0824716981; Lloyd, The Art, Science and Technology of Pharmaceutical Compounding (1999); and Pickar, Dosage Calculations (1999)). As is known in the art, adjustments for patient condition, systemic versus localized delivery, as well as the age, body weight, general health, sex, diet, time of administration, drug interaction and the severity of the condition may be necessary, and will be ascertainable with routine experimentation by those skilled in the art.

The administration of the agents can be done in a variety of ways as discussed above, including, but not limited to, orally, subcutaneously, intravenously, intranasally, transdermally, intraperitoneally, intramuscularly, intrapulmonary, vaginally, rectally, or intraocularly.

In one embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly useful are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that compositions of the invention when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

The compositions for administration will commonly comprise an antibody dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

The compositions containing immunosuppressive agents, e.g. antibodies, etc. can be administered for therapeutic treatment. Compositions are administered to a patient in an amount sufficient to immunosuppress the patient, as described above. An amount adequate to accomplish this is defined as a “therapeutically effective dose.” Single or multiple administrations of the compositions may be administered depending on the dosage and frequency as required and tolerated by the patient. The particular dose required for a treatment will depend upon the medical condition and history of the mammal, as well as other factors such as age, weight, gender, administration route, efficiency, etc.

In the methods of the invention, the agents are administered as a short course of therapy prior to transplantation. Usually the treatment is completed at least about two weeks prior to transplantation, at least one week prior to transplantation, at least about 5 days prior to transplantation, at least about 3 days prior to transplantation. The process may be repeated if necessary, e.g. may be repeated twice, three times, four times, five times, or more, as necessary.

### Conditions for Treatment

The indications for stem cell transplantation vary according to disease categories and are influenced by factors such as cytogenetic abnormalities, response to prior therapy, patient age and performance status, disease status (remission vs relapse), disease-specific prognostic factors, availability of a suitable graft source, time of referral, and time to transplant.

Autologous HSCT is currently used to treat a number of conditions, including without limitation multiple myeloma, non-Hodgkin lymphoma, Hodgkin disease, neuroblastoma, germ cell tumors, autoimmune disorders such as systemic lupus erythematosus (SLE), systemic sclerosis, Amyloidosis, and the like. Additional disorders are suitable for such treatment when the autologous cells are genetically corrected.

Allogenic HSCT is currently used to treat the following disorders: Acute myeloid leukemia, Acute lymphoblastic leukemia, Chronic myeloid leukemia; Chronic lymphocytic leukemia, Myeloproliferative disorders, Myelodysplastic syndromes, Multiple myeloma, Non-Hodgkin lymphoma, Hodgkin disease, Aplastic anemia, Pure red cell aplasia, Paroxysmal nocturnal hemoglobinuria, Fanconi anemia, Thalassemia major, Sickle cell anemia, Severe combined immunodeficiency (SCID), Wiskott-Aldrich syndrome, Hemophagocytic lymphohistiocytosis (HLH), Inborn errors of metabolism—e.g., mucopolysaccharidosis, Gaucher disease, metachromatic leukodystrophies, and adrenoleukodystrophies, Epidermolysis bullosa, Severe congenital neutropenia, Shwachman-Diamond syndrome, Diamond-Blackfan anemia, Leukocyte adhesion deficiency, and the like.

In some embodiments a patient is treated for an anemia in which endogenous HSC are at a growth disadvantage compared to wild-type, genetically corrected or otherwise modified HSC. In some embodiments the patient has Fanconi Anemia. Fanconi anemia (FA) is a rare genetic disorder, in the category of inherited bone marrow failure syndromes. There are several subtypes of FA that result from the inheritance of two gene mutations in each of at least 23 different genes. Most of the subtypes share the characteristic symptoms and findings. The disorder is often associated with a progressive deficiency of all bone marrow production of blood cells, red blood cells, white blood cells, and platelets. Affected individuals have an increased risk of developing acute myeloid leukemia (AML), or tumors of the head, neck, skin, gastrointestinal system, or genital tract. It is usually inherited as an autosomal recessive genetic disorder, but X-linked inheritance has also been reported.

The symptoms of FA vary from person to person. Identified symptoms include a variety of physical abnormalities, bone marrow failure, and an increased risk of malignancy. Physical abnormalities normally reveal themselves in early childhood, but in rare cases diagnoses are made in adulthood. Blood production problems often develop between 6 to 8 years of age. Bone marrow failure eventually occurs in the majority of affected individuals, although the progression and age of onset vary. Patients who live into adulthood are likely to develop head and neck, gynecologic, and/or gastrointestinal cancer at a much earlier age than the general population, whether or not they had earlier blood problems.

Progressive bone marrow failure typically presents by the age of 10 and is usually accompanied with low hemoglobin, low platelet levels or low white blood cells. By age 40 to 50 years, the estimated incidence of bone marrow failure as the first serious event is more than 90%. Affected individuals develop low levels of all the cellular elements of the bone marrow which can lead to anemia, leukopenia, neutropenia, and thrombocytopenia.

Individuals with FA have a higher risk than the general population of developing certain forms of cancer including acute myeloid leukemia and specific solid tumors. Affected individuals may are at extremely high risk of developing cancer affecting the head and neck region, gastrointestinal tract, esophagus or gynecologic regions. Most of these are a specific form of cancer, known as squamous cell carcinoma. FA patients whose bone marrow failure is treated with androgens have in increased risk of liver tumors. FA patients whose bone marrow failure is treated with genotoxic agents and HSCT have an increased risk of various solid tumors.

Mutations in at least 23 genes can cause FA. The proteins encoded by these genes are in the FA pathway, which is involved in DNA repair. Eight proteins form a complex known as the FA core complex, which activates two genes to make FANCD2 and FANCI. The activation of these two proteins brings DNA repair proteins to the area of DNA damage. Seventy to 80 percent of cases of FA are due to mutations in one of three genes, FANCA, FANCC, and FANCG, relating to the FA core complex. Mutations in the following genes also cause FA and are inherited in an autosomal recessive manner: BRCA2, BRIP1, FANCB, FANCD2, FANCE, FANCF, FANCI, ERCC4, FANCL, FANCM, PALB2, RAD51C, SLX4, and UBE2T. The FANCB gene is located on the X chromosome, and causes less than 1 percent of all cases of FA. This FA gene is inherited as an X-linked recessive trait. Mutations in the RAD51 gene cause autosomal dominant FA.

Molecular genetic testing is available for all 23 genes associated with FA. Complementation testing can be done in order to identify which FA gene is mutated. Additional sequence analysis of the appropriate gene can be done to determine the specific mutation in that gene. If a mutation is not identified, deletion/duplication analysis is available clinically for the genes associated with FA.

### Experimental

In this study, we explored the use of antagonistic αCD117 mAb alone and in comparison with αCD117+CD47 mAb and CD117-ADC in the FA setting using Fancd2^{-/-} mice assessing for host HSC depletion, enhanced donor chimerism post HSCT and tissue toxicities. Additionally, we explored the use of these agents in combination with αCD4, αCD8, αCD40L and αCD122 mAb or rapamycin combination immunosuppression treatment using C57BL/6J and C57BL/6J and BALB/cAnNCr/F1 generation donors representing HLA-matched and haploidentical MHC HSCT. Fancd2⁷ mice were selected for these studies given their use in prior published reports and their more pronounced hematopoietic phenotype compared to alternative available FA mouse models.

### Results

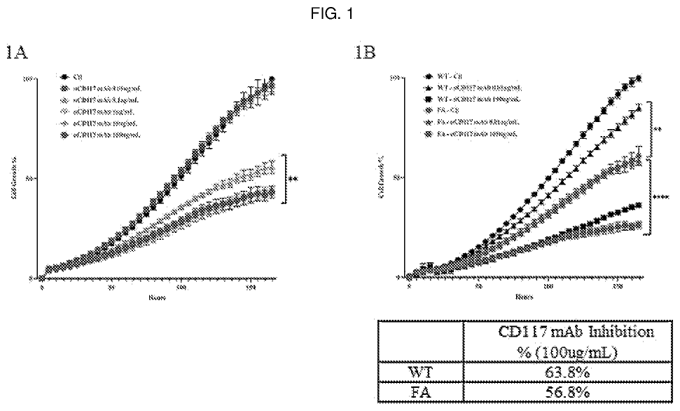

Naked Antagonistic αCD117 mAb suppresses growth rate of FA HSPCs in vitro. We first determined the in vitro potency and growth effect of naked antagonistic αCD117 mAb on hematopoietic and progenitor cells (HSPCs) which were isolated from FA (Fancd2^{-/-}) and wildtype (WT) bone marrow via fluorescence-activated cell sorting (FACS) on cells of the Lineage⁻Sca-1⁺CD150⁺CD48⁻CD34⁻CD135⁻CD244⁻ phenotype. As αCD117 mAb was used in cell culture, CD117 was not included for phenotypic identification to minimize possible confounding results of competing CD117 mAbs used for isolation. We found that FA HSPC growth was restricted by αCD117 mAb exposure in a dose dependent manner and reached steady-state growth inhibition at 1 μg/ml and higher (FIG. 1A). To examine the comparative effect of αCD117 mAb on FA and WT HSPCs, overall HSPC growth rate was observed based on untreated WT HSPC controls over a 6-day period. Unsurprisingly, FA HSPCs displayed a slower proliferation rate in both untreated and αCD117 mAb treated groups in comparison to WT HSPCs (FIG. 1B). However, HSPCs in both strains exhibited similar growth inhibition post-αCD117 mAb treatment, with 63.8% and 56.8% in WT and FA HSPCs, respectively. There were no enhanced or suppressed activities observed for FA HSPCs compared to WT HSPCs in the presence of αCD117 mAb, which suggests that the mechanistic function of αCD117 mAb is not dependent underlying hematopoietic condition.

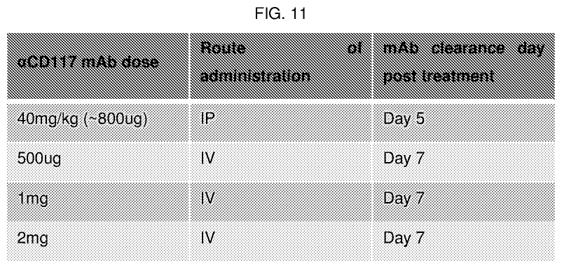

Minimal effects with naked antagonistic αCD117 mAb in vivo in FA mice. To address whether HSC depletion in vivo mimics the dose-dependent results of naked antagonistic αCD117 mAb observed in vitro, we investigated the effect of various concentrations of αCD117 mAb in the FA (Fancd2^{-/-}) mouse model. We tested four different concentrations of αCD117 mAb (40 mg/kg, 500 ug, 1 mg, and 2 mg) and two different routes of administration (intravenous—IV and intraperitoneal—IP) based on prior reports (Czechowicz et al., 2007; Xue et al., 2010; Chandrakasan et al., 2017). Prior to studying the effects of αCD117 mAb in bone marrow (BM), it was necessary to examine the clearance profile of αCD117 mAb in peripheral blood (PB) serum to determine a suitable timeframe for HSC depletion assessment. Clearance of αCD117 mAb in the serum was observed in all animals between 5-7 days post-αCD117 mAb administration (FIG. 11), and prolonged clearance did not occur when animals were given a higher dose of αCD117 mAb.

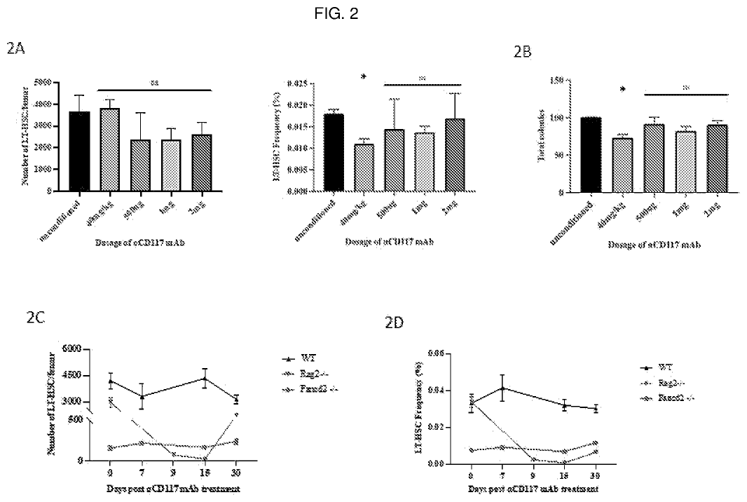

Next, we studied the effect of naked αCD117 mAb on bone marrow composition. Bone marrow was harvested according to the corresponding serum clearance day of αCD117 mAb per administered dose. Surprisingly, there were no significant differences in HSC absolute number, frequency, nor functionality despite testing many different dosages of αCD117 mAb and routes of administration (FIG. 2A-B) including those previously reported to cause HSPC depletion in this model (40 mg/kg IP). Given this result, we decided to continue this study with 500 μg of αCD117 mAb IV to compare with our prior reports of αCD117 mAb showing efficacy in the Rag2^{-/-} SCID setting. With 500 μg of αCD117 mAb injection via IV, the absolute number and frequency of HSCs in Fancd2^{-/-} and WT mice were not affected and remained stable for the following 30 days (FIG. 2C-D). In contrast, as previously reported HSCs in immunodeficient Rag2^{-/-} SCID mice were strongly affected by αCD117 mAb and transiently depleted on the day of serum clearance (day 9) and recovered by day 30. Our results for SCID and WT mice were consistent with prior reports; however, the results obtained in this study for Fancd2-contradicted previously reported HSC depletion 7 days after αCD117 mAb administration.

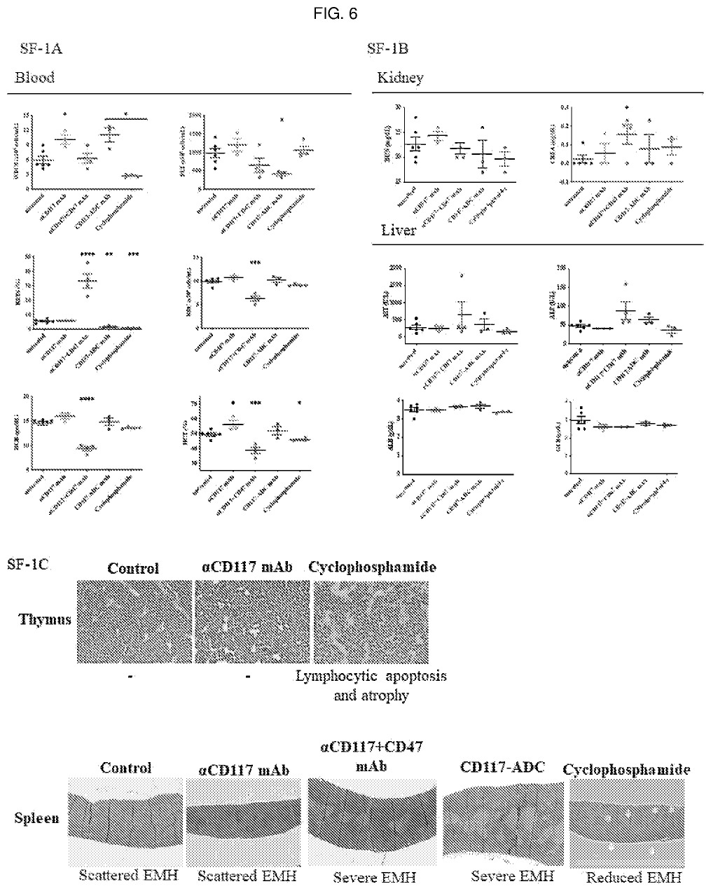

Different safety and efficacy profiles with αCD117 mAb conditioning strategies in FA mice. Given that multiple alternative regimens for HSCT conditioning using monoclonal antibodies against CD117 have shown efficacy in different pre-clinical models, with parallel clinical agents in development of each, we sought to explore the safety and efficacy profiles of each in FA mice to determine the optimal conditioning regimen. To determine the safety of each agent, we treated FA mice with antagonistic αCD117 mAb, αCD117+CD47 mAb and CD117-ADC. Animals were monitored for 7 days and minimal toxicities were observed with each agent in the blood count for blood, kidney, and liver (FIG. 6A-B). αCD47 mAb conditioned animals exhibited severe anemia without pre-dosing of mAb. Toxicities were minimized with pre-dosing of 100 μg of αCD47 mAb. Furthermore, no toxicities were found in H&E staining from thymus and spleen compared to the positive control animal conditioned with cyclophosphamide (FIG. 6C). Notably no toxicities of any kind were observed with αCD117 mAb alone.

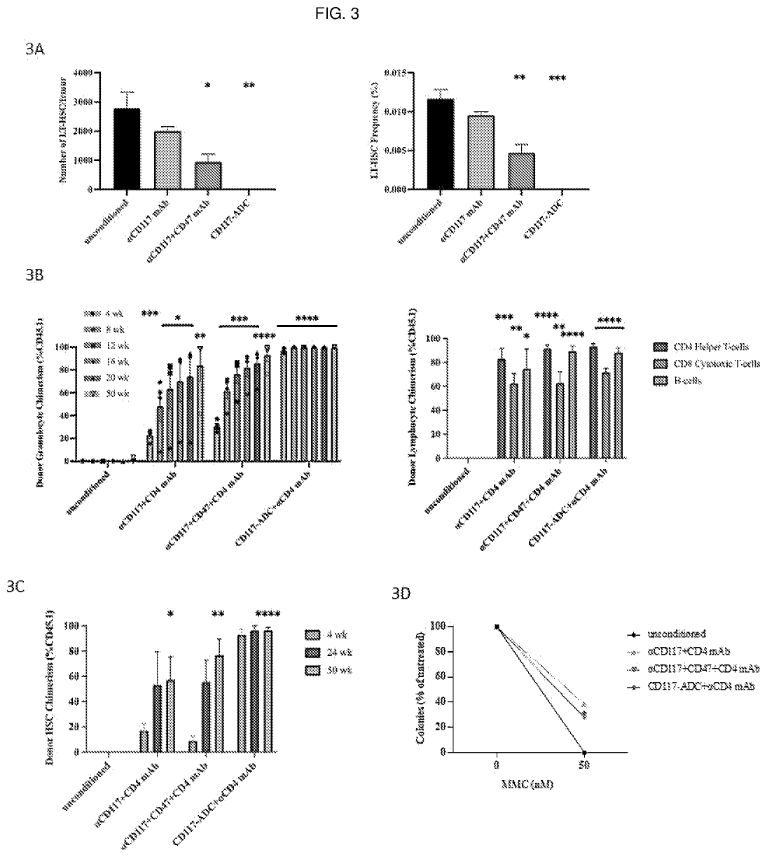

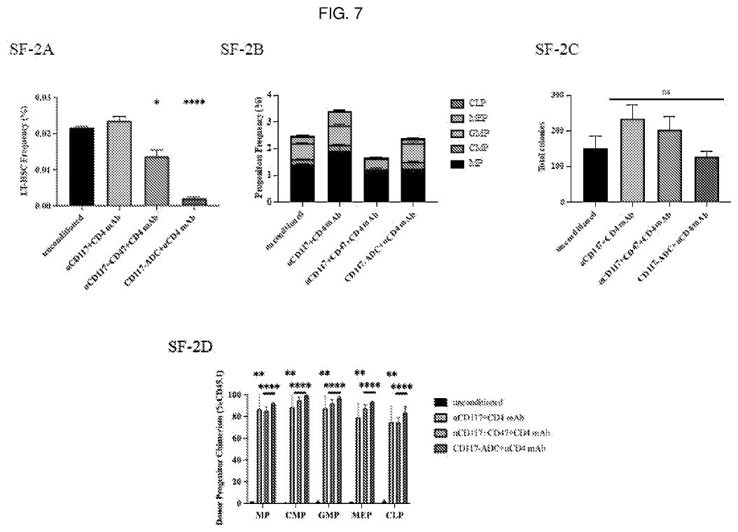

To determine the efficacy of each approach for HSC depletion in BM, FA animals were treated with each agent and BM was assessed at 7 days post-conditioning. HSCs were only observed to be near completely depleted in animals conditioned with CD117-ADC, whereas combination αCD117+CD47 mAb depleted HSCs to only ˜50% reduction rate and αCD117 mAb alone was insufficient to significantly deplete HSCs in treated animals (FIG. 3A). Next, we tested the efficacy of each conditioning regimen to enable during engraftment upon HSCT with whole bone marrow (WBM) from allogeneic donors. Given the reported slight mismatch in the genetic background between WT C57BL/6J donor cells and Fancd2^{-/-} mouse model representative of HLA-matched HSCT, we similarly included CD4 T-cell depleting αCD4 mAb as an additional conditioning agent to minimize immunological barriers between the two strains. We assessed the HSC frequency 7-days after combined conditioning and there was no difference in depletion of HSCs and BM progenitors in the animals which received the addition of αCD4 mAb (FIG. 7A-C).

Importantly, donor chimerism was observed in all cell types assessed in the peripheral blood (PB) with each of the different conditioning regimens 50 weeks after HLA-matched WBM transplantation (FIG. 3B). The most profound donor engraftment in PB was observed in animals conditioned with CD117-ADC where extremely high donor chimerism (>95%) was observed as early as 4 weeks after WBM transplantation. Although the αCD117+CD4 mAb and combination αCD117+CD47+CD4 mAb treatments did not display high donor engraftment at early timepoints, donor engraftment in granulocytes steadily grew up to >80% over 50 weeks post-transplantation. We also determined HSC donor chimerism throughout the experiment at 4 weeks, 24 weeks, and 50 weeks after transplantation by BM aspirate. The resulting donor HSC and progenitor chimerism was similar to the donor chimerism observed in PB, in which donor chimerism was the most profound in the CD117-ADC treated group and steadily increased in αCD117+CD4 mAb and αCD117+CD47+CD4 mAb treated groups. (FIG. 3C; FIG. 7D). Given FA cells are known to have impaired HSC proliferative capacity and defective DNA repair with sensitivity to DNA damaging agents such as mitomycin-C (MMC), HSC functionality was examined with colony forming cell (CFC) with and without MMC at the end of study. All conditioned and transplanted groups showed higher colony-formation with the presence of 10 nM MMC compared to the unconditioned group with no colonies detected in the culture (FIG. 3D) indicative of functional disease correction.

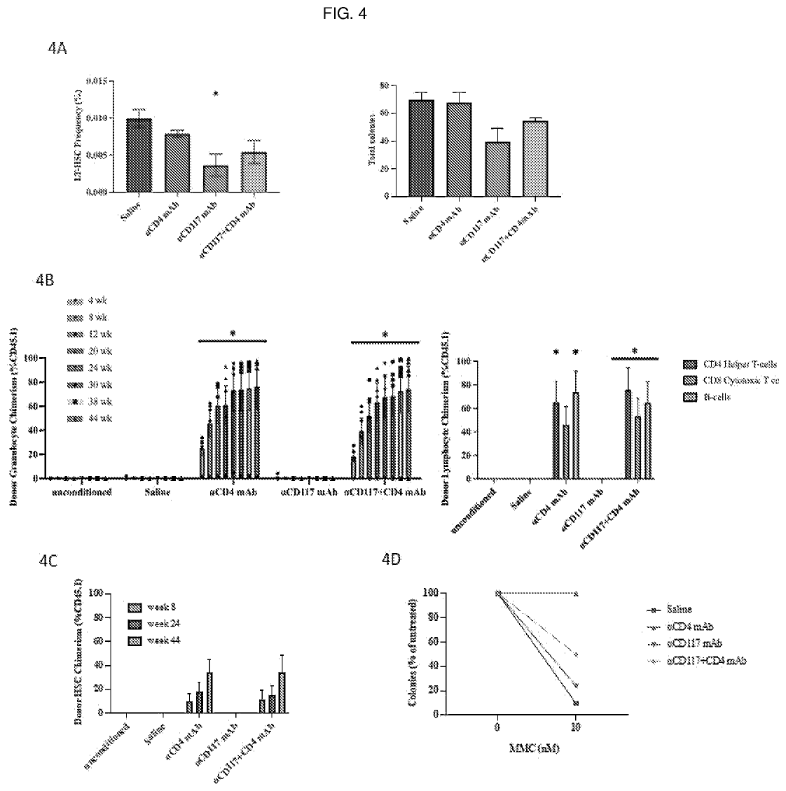

Immunosuppression alone enables high donor engraftment post HLA-matched HSCT in FA mice. Next, we sought to explore if the use of CD4-T-cell depleting αCD4 mAb alone would enhance donor chimerism in FA mice without HSC-targeting antibodies given the high donor engraftment that was observed post αCD117+CD4 mAb conditioning despite lack of HSC depletion of the αCD117 mAb. To explore this, animals were conditioned with αCD4 mAb alone, αCD117 mAb alone or with the αCD117+CD4 mAb combination as was previously shown to be effective. BM assessment was performed 7 days after initial conditioning and all groups showed minimal depletion of phenotypic or functional HSCs (FIG. 4A). Surprisingly, despite lack of HSC depletion in the BM before transplantation, αCD4 mAb alone and combination αCD117+CD4 mAb conditioned groups both exhibited high donor engraftment in granulocytes and other lymphocyte lineages to >80% by 44 weeks after HLA-matched HSCT using donor WT C57BL/6J WBM cells (FIG. 4B). Furthermore, HSC donor chimerism was also detected in both the αCD4 mAb alone and combination αCD117+CD4 mAb groups and the level of donor chimerism increased steadily over time from 10% to 40% (FIG. 4C) likely due to a competitive advantage of the donor WT cells to host FA cells. HSCs also showed higher resistance toward MMC in both the αCD4 mAb alone and combination αCD117+CD4 mAb conditioned and transplanted animals compared to the animals transplanted with saline only conditioning, which is consistent with the data obtained from HSC donor chimerism. Notably, the FA mice conditioned with αCD117 mAb alone had no donor chimerism observed in the peripheral blood or in the bone marrow.

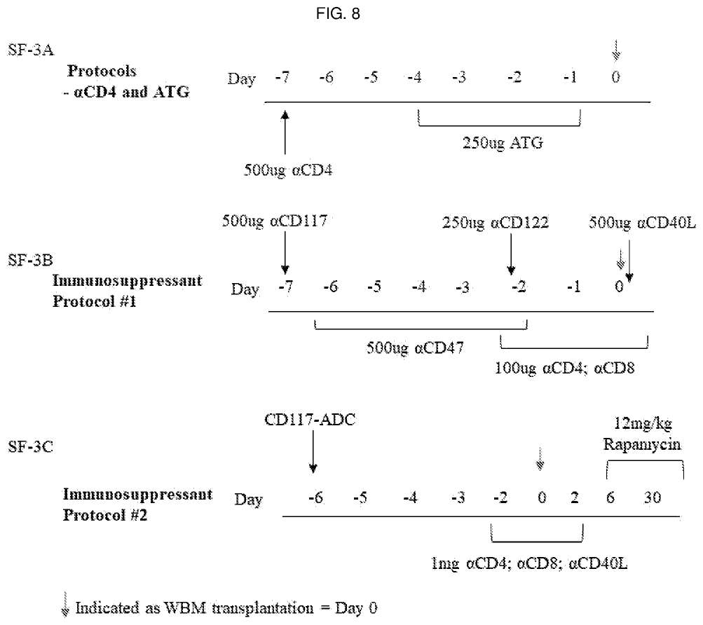

Immunosuppression cocktails enable high donor engraftment even post haploidentical HSCT in FA mice. Based on the observation that CD4 T-cell depleting αCD4 mAb conditioning alone enabled high donor chimerism post HLA-matched transplantation in FA mice, we further explored whether immunosuppression alone could allow for similar efficacy across larger immune barriers enabling use of more mismatched donors. To determine this, based upon prior literature we tested four different conditioning regimens with αCD4 mAb alone, αCD4 mAb+mouse αthymocyte globulin (ATG), combination αCD4, αCD8, αCD40L, and αCD122 mAbs (Immunosuppressant #1); and combination αCD4, αCD8, αCD40L, and rapamycin (Immunosuppressant #2). The conditioning agents were administered at varying timepoints as previously reported and donor haploidentical (C57BL/6J and BALB/cAnNCrl F1 generation) WBM cells were transplanted seven days after initial conditioning (FIG. 8).

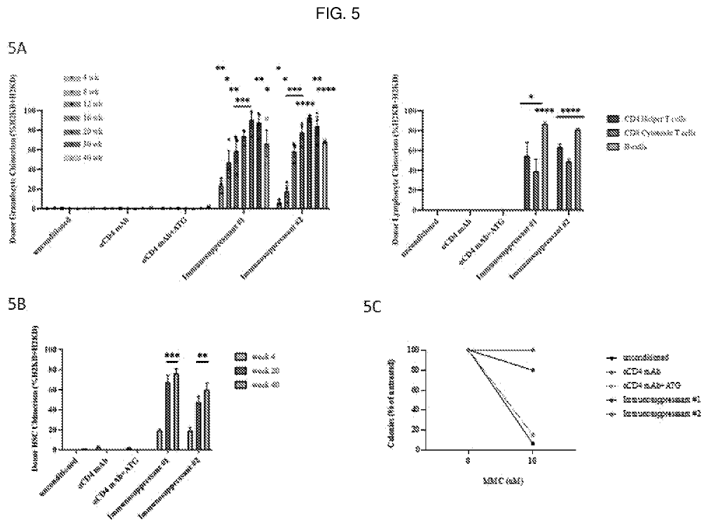

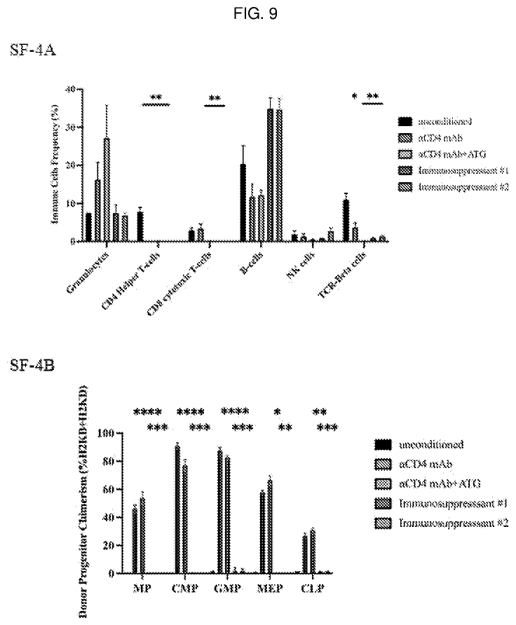

To determine the extent of immune ablation with each regimen, we examined the peripheral blood for mature immune cells before haploidentical WBM transplantation (FIG. 9A). Only subsets of T-cells were affected and depleted in all of the conditioned groups, including helper T-cells, cytotoxic T-cells, and TCR-beta T-cells, while B cells and NK cells were largely not affected with any of the regimens. Although the evaluation of immunological effects of all of the conditioned groups were similar, only Immunosuppressant #1 and Immunosuppressant #2 were able to enable high donor multi-lineage PB and BM HSC engraftment in the haploidentical setting, averaging 90% and 70% respectively 40 weeks after transplantation, whereas αCD4 mAb and combination αCD4 mAb+ATG were not capable of enabling donor engraftment (FIG. 5A-B). High donor engraftment post haploidentical WBM HSCT was also observed in the BM progenitors MP, CMP, GMP, MEP, and CLP populations (FIG. 9B) in the Immunosuppressant #1 group and Immunosuppressant #2 conditioned mice. MMC resistance of HSC correlated with donor engraftment (FIG. 5C) and only Immunosuppressant #1 and Immunosuppressant #2 conditioned mice showed higher significant MMC resistance in the functionally assay compared to the unconditioned group.

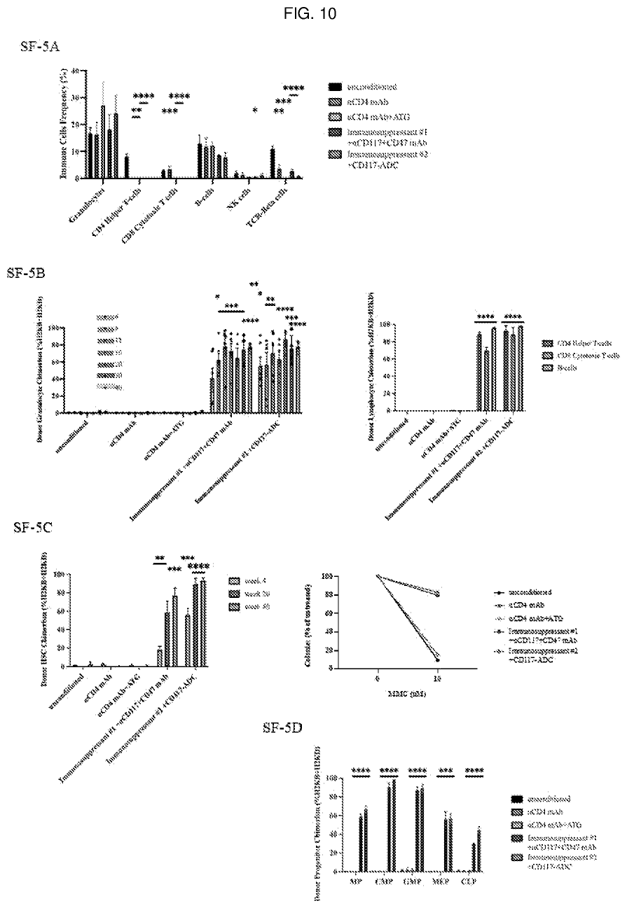

We then added HSC depleting agents with the immunosuppressant cocktail to study if the combination would enhance donor chimerism further, including αCD117 mAb, αCD47 mAb, and CD117-ADC. We examined immunological effects of mature immune cells in the peripheral blood and the result was consistent with immunosuppressant cocktail only groups, that only subsets of T cells were affected (FIG. 10A). With the addition of αCD117+CD47 mAb and CD117-ADC, the initial donor chimerism at 4 weeks after mismatched WBM transplantation were higher at an average of 40% and 60%. However, donor chimerism in the peripheral blood, lineage, and BM progenitors did not increase further in the long-term and the level of donor chimerism was similar to immunosuppressant cocktail regimens (FIG. 10B, D). Only HSC donor chimerism was enhanced to >90% in Immunosuppressant #2+CD117-ADC group, which was also reflected in the functionality assay with MMC resistance (FIG. 10C).

Based on the observation that CD4 T cell-depleting αCD4 mAb conditioning alone enabled high donor chimerism after HLA-matched transplantation in Fancd2^{-/-} mice, we further explored whether immunosuppression alone could allow for similar efficacy across larger immune barriers, enabling the use of more mismatched donors. To determine this we tested 4 different conditioning regimens: CD4 mAb alone; αCD4 mAb plus mouse anti-thymocyte globulin (ATG); a combination αCD4, αCD8, αCD40L, and αCD122 mAbs (immunosuppressant 1); and a combination of αCD4, αCD8, and αCD40L mAbs and rapamycin (immunosuppressant 2). The conditioning agents were administered at varying doses and time points as reported previously, and donor haploidentical WBM cells from C57BL/6N×Balb/c F1 mice underwent transplantation at 7 days after initial conditioning.

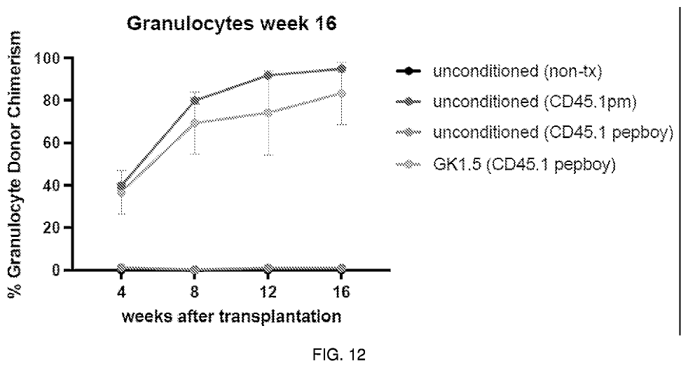

To determine the extent of immune ablation caused by each regimen, we assessed for mature immune cells in PB before haploidentical WBM transplantation. Subsets of T cells were depleted in all conditioned groups, including helper T cells, cytotoxic T cells, and TCR-β T cells; however, B cells and NK cells were largely unaffected by any of the regimens. Although the immunologic effects of each conditioning regimen were similar in our evaluation, only immunosuppressant 1 and 2 enabled high donor multilineage PB and BM HSC engraftment in the haploidentical setting, with ˜60% to ˜80% donor chimerism at 40 weeks post-transplantation, whereas αCD4 mAb alone and the combination of αCD4 mAb and ATG did not enable donor engraftment (FIG. 12). This may be related to the differences in immune responsiveness after CD40L administration, which might not be evident on gross assessment of lymphocyte numbers; this was included in both the immunosuppressant 1 and 2 regimens. High donor engraftment after haploidentical WBM HSCT also was observed in the BM progenitors, including myeloid progenitors, common myeloid progenitors, granulocyte-monocyte progenitors, megakaryocytic-erythroid progenitors, and common lymphoid progenitors, in immunosuppressant 1- and 2-conditioned mice only. MMC resistance of HSPCs in CFC assays correlated with donor engraftment, and only immunosuppressant 1- and 2-conditioned mice showed significantly higher MMC resistance compared with the unconditioned group.

We then tested the addition of the HSC-targeted agents to the immunosuppressant regimen cocktails to determine whether the combination with αCD117+αCD47 mAb or CD117-ADC would continue to enable or further enhance donor chimerism. The results of assessment for mature immune cells in PB were consistent with findings in immunosuppressant cocktail alone groups, with profound depletion only of T cell subsets. The addition of αCD117+CD47 mAb and CD117-ADC produced greater initial donor chimerism at 4 weeks after mismatched haploidentical WBM transplantation, at an average of ˜40% and ˜55%, respectively. However, donor chimerism in PB and BM showed no further increases, and the degrees of long-term donor chimerism and CFC resistance were similar to those seen with the immunosuppressant regimens alone. Only HSC donor chimerism was enhanced to >90% in the immunosuppressant 2 plus CD117-ADC group.

Fanconi Anemia is a challenging disease to treat due to the inherent genetic defects in patients, which cause problems in DNA repair that make current treatments for bone marrow failure and malignancies more difficult to utilize in this setting. Specifically, allogeneic stem cell transplants and other cancer treatments currently utilize genotoxic agents such as alkylating chemotherapy+/−irradiation, which have increased short-term and long-term side effects in this patient population including high rates of subsequent malignancies. We have previously shown that antagonistic αCD117 mAb and CD117-ADCs can robustly deplete host HSCs and enable high donor hematopoietic stem and progenitor cell engraftment post HSCT in various settings, eliminating the need for commonly used genotoxic agents, which would be particularly beneficial in the FA setting. Given these results, parallel clinical agents are now in advancing development creating opportunities to use in FA patients. However, while the pre-clinical anti-mouse agents have been applied in preliminary experiments in FA mouse models, full efficacy and safety has not been determined, nor have these approaches been compared head-to-head, making it challenging to determine if and which of these clinical agents should be used in FA patients. Furthermore, these approaches have not been applied in true allogenic settings with combined immunosuppression which is critical to prevent immunologic graft rejection.

Importantly, through the studies performed we found that each αCD117 mAb agent was well-tolerated in the FA setting without increased toxicity, as compared to previous reports of these agents in wild-type mice. Blood counts, chemistries and tissue histology were all improved compared to chemotherapy and irradiation controls, however differences in side-effects were observed with combination antagonistic αCD117+CD47 mAb resulting in mild cytopenia and CD117-ADC resulting in mild elevation in transaminases.

However, larger differences in the various agents were found when assessing effects on hematopoietic stem and progenitor cells (HSPCs). Interestingly, we found that antagonistic αCD117 mAb treatment alone was capable of inhibiting Fancd2^{-/-} HSPCs growth in vitro, however contradicting prior reports it was insufficient to robustly deplete HSCs in vivo in the FA setting. However, with the addition of αCD47 mAb, MIAP410, or the use of CD117-ADC, in vivo HSC depletion was achieved. Surprisingly, while treatment with αCD4 mAb alone did not cause significant HSC depletion, it was able effectively enable significant donor chimerism post SCT in the minor mismatched setting which suggests that immunosuppression alone is sufficient to enable hemopoietic cell transplantation in FA settings. Furthermore, serial bone marrow aspirates showed increasing donor HSC chimerism over time which is likely due to the competitive advantage of wildtype HSCs over FA HSCs which could explain why immunosuppression alone is uniquely sufficient in this disease setting.

Based on the result that HSC depletion is not necessary for effective HLA-matched BMT, we further hypothesized that robust immunoablation-only conditioning may be effective in enabling donor chimerism in FA HLA-mismatched settings. Excitingly this effect was indeed observed in the haplo-identical HLA-mismatched setting where allogeneic SCT engraftment was obtained by utilizing only a robust immunosuppressant regimen as conditioning, which does not cause host HSC depletion with resulting disease susceptibility, as assessed by bone marrow resistance to DNA-damaging agent MMC. Our studies demonstrated competitive advantage of donor cells for HSC restoration and HSCT efficacy with use of an immunosuppressant antibody-only conditioning regimen in both HLA-matched and mismatched MHC HSCT settings.

This approach has many advantages, the largest being the elimination of need for use of an additional potentially expensive agent. In addition, unlike HSC-targeted antibodies, immunosuppressant agents do not have concerns regarding graft depletion without careful monitoring of antibody clearance and can eliminate any uncertainty of antibody clearance in each patient before infusion of HSCT.

Although the immunosuppression cocktails used in these studies are not directly clinically translatable at this time due to lack of parallel αCD4, αCD8, αCD40L and αCD122 clinical agents, these studies suggest that alternative immunosuppressant regimens will enable the same effect. Given that anti-thymocyte globulin (ATG) and depleting αCD52 mAbs have both mouse and human targeting agents available, we tried the pre-clinical agents in our model however these were insufficient to enable donor engraftment in the HLA-mismatched setting suggesting a need for more robust immune ablation.

It is predicted that current clinically used immunoablation protocols of ATG, Rituximab, Cyclophosphamide and Fludarabine would be sufficient, however this is difficult to model in mice due to lack of readily available pre-clinical mouse agents. Furthermore, while this regimen would abrogate the need for use of total body irradiation or busulfan, it still contains chemotherapy and further highlights the needs for improved immune ablation which potentially could be obtained with regents such as αCD2 and αCD3 mAbs that are currently in clinical development.

In conclusion, our discovery that immune ablation is sufficient conditioning for successful allogenic FA HSCT is important for a number of reasons: 1) it shows that genotoxic HSC-depleting agents, such as alkylating chemotherapy and irradiation, are not necessary for effective immunoablation, 2) it shows that while αCD117 mAb agents may enable higher donor chimerism when added to immunoablation chemotherapy, they may not be necessary, and 3) that in settings where immunologic rejection is not possible (e.g. in autologous gene therapy and in utero HSCT), HSC-depleting agents may not be necessary for efficacious HSCT due to competitive advantage of the transplanted cells relative to the endogenous HSC. Given that αCD117 mAb conditioning agents are in advancing clinical development, these studies are of high importance. Additionally, these studies justify the development of immune-ablation only protocols in FA.

### Methods

In vitro liquid culture of mouse HSPCs. In vitro liquid culture of Fancd2−/− and C57BL/6N mouse HSPCs (Lin⁻Sca-1⁺CD150⁺CD48⁻CD34⁻CD135⁻CD244⁻) were performed to assess for αCD117 mAb potency in vitro. Briefly, 200 HSPCS were sorted by BD FACS Aria II (BD Biosciences, San Jose, CA) into individual well of a 96-well round-bottom plate. Each well contained 100 μL of F12 media supplemented with 10 mM HEPES, 1×P/S/G, 1×ITSX, 1 mg/mL PVA, 100 ng/mL TPO, and 10 ng/mL SCF. Sorted HSPCs were then treated with varying concentrations (0.01 ug/mL, 0.1 ug/mL, 1 μg/mL, 10 ug/mL, 100 ug/mL) of antagonistic αCD117 mAb (Bio X cell, Lebanon, NH) immediately thereafter. Viable cells were imaged under IncuCyte live cells analysis program for 7 days.

Mice. All animal housing and procedures were approved by the International Animal Care and Use Committee (IACUC) at Stanford University Committee. C57BL original derived with 129/SvJ background. Fanconi Complementation Group D2 knockout (Fancd2^{-/-}) mice were generous gift from Dr. Kenneth Weinberg, Stanford University, Stanford, CA. Wild-type (B6.SJL-Ptprc^{a}Pepc^{b}/Boy) and Balb/c (BALB/cAnNCrl) animals were purchased from Charles River to interbreed for all HLA mismatched transplantation experiments. All animals were aged between 8-12 weeks at time for all studies.

Antibody and conditioning reagents administration. All monoclonal antibodies were prepared with sterile PBS for all dilution. 2 mg/mL of Benadryl (Sigma-Aldrich) was injected inter-peritoneally (IP) 15 minutes before injection of antagonistic αCD117 mAb on day 0 intravenously (IV). 100 ug of αCD47 mAb (Bio X Cell, Lebanon, NH) was administered as a priming dose prior to 5 consecutive days (IP) injections of 500 μg for all transplantation studies where it was included. CD117-ADC was prepared and diluted to the concentration of 1.5 mg/kg per animal via IV administration route. All immunoablation antibodies for HLA-mismatched studies were purchased from Bio X Cell and administrated accordingly to the schema in FIG. 8.

Antibody serum clearance in FA mice. αCD117 mAb were prepared and administered as described previously. 50 μL of peripheral blood were collected and serum were isolated every other day after administration. Peripheral serum was used to stain the CD117 expressing P185 murine cell line with the addition of Fc-blocker (CD16/32). The signal of αCD117 mAb was detected using a goat-anti-rat secondary antibody by flow cytometry analysis.

BM transplantation and peripheral blood (PB) chimerism. BM cells were harvested from donor mice and 20 million whole bone marrow cells were transplanted retro-orbitally into each recipient mice with various antibody conditioning treatments. All peripheral blood samples were collected retro-orbitally into 0.5 mM EDTA for PB donor chimerism evaluation every 4 weeks. RBCs were removed with RBC lysis buffer (eBioscience, San Diego, CA) and donor chimerism was evaluated in B-cells (Ter119⁻CD3⁻B220⁺CD19⁺), Cytotoxic T-cells (Ter119⁻CD3⁺B220⁻CD8⁺CD4⁻), Helper T-cells (Ter119⁻CD3⁺B220⁻CD8⁻CD4⁺), and Granulocytes (Ter119⁻CD3⁻B220⁻CD11b⁺Gr-1⁺). All antibodies for PB analysis were purchased from Biolegend, San Diego, CA. All the samples were acquired from BD FACSAria II at Institute for Stem Cell Biology and Regenerative Medicine FACS Core at Stanford University and analyzed by Flowjo software.

BM analysis for depletion and chimerism. Bone marrow (BM) HSPC depletion was measured from BM aspirates from the femur on the day of transplantation prior to BM transplantation. BM HSPC donor chimerism was evaluated by BM aspirate at differing timepoints as indicated and at time of necropsy 40-50 weeks after transplantation. RBCs were removed with RBC lysis buffer and assessed for donor chimerism in HSC (Lineage⁻Sca-1⁺c-Kit⁺CD150⁺CD48⁻), Myeloid Progenitor (Lineage⁻Sca-1⁻c-Kit⁺IL7Ra⁻), CMP (Lineage⁻Sca-1⁻c-Kit1⁺IL7Ra⁻CD34⁺CD16/32⁻), MEP (Lineage⁻Sca-1⁻c-Kit⁺IL7Ra⁻CD34⁻CD16/32⁻), GMP (Lineage⁻Sca-1⁻c-Kit⁺IL7Ra⁻CD34⁺CD16/32⁺), and CLP (Lineage⁻Sca-1⁺c-Kit⁺IL7ra⁺). All antibodies for BM chimerism analysis were purchased from BioLegend, San Diego, CA. All the samples were acquired and analyzed the same way as PB chimerism listed above.

Colony formation cell assay. Single cell suspension of BM cells was obtained from aspiration or necropsy with removal of RBCs. Cells were suspended in IMDM and plated triplicate for methylcellulose culture in methocult M3434 media (StemCell Technologies, Vancouver, Canada) with and without 10 nM mitomycin-C (MMC) (Millipore Sigma, Burlington, MA). After 7 days of culture, colonies in methocellulose culture were evaluated and analyzed by StemVision (StemCell Technologies, Vancouver, Canada).

Toxicity assessment. Antibody treatment was administered as described earlier and Cyclophosphamide treatment group was served as a positive control for toxicity measurement. Bone marrow, thymus, spleen, liver, stomach, and small intestine/large intestine were harvested 7 days after antibody treatment and placed in 4% paraformaldehyde for >1 day. All organs were paraffin-embedded, sectioned, stained with hematoxylin and eosin, and mounted using xylene-based media by the Department of Comparative Medicine's Diagnostic Laboratory at Stanford University. Histology images were assessed under light microscopy. In addition, peripheral blood and serum were submitted to Stanford Diagnostic Lab for toxicity evaluation.

Statistics. All data was analyzed using GraphPad Prism version 9.10. All significance data was analyzed with unpaired t-tests and non-parametric tests (*) P<0.05; (**) P<0.01; (***) P<0.001; (****) P<0.0001. The statistical data are presented as standard error of the mean (SEM).
Velleuer E, Carlberg C. Impact of epigenetics on complications of fanconi anemia: The role of vitamin d-modulated immunity. Nutrients. 2020; 12(5).
Soulier J. Fanconi Anemia. Hematology. 2011; 2011(1):492-497.
Kitao H, Takata M. Fanconi anemia: A disorder defective in the DNA damage response. Int. J. Hematol. 2011; 93(4):417-424.
Auerbach A D. Fanconi anemia and its diagnosis. Mutat. Res.—Fundam. Mol. Mech. Mutagen. 2009; 668(1-2):4-10.
Mathew C G. Fanconi anaemia genes and susceptibility to cancer. Oncogene. 2006; 25(43):5875-5884.
Alter B P, Giri N. Thinking of VACTERL-H? Rule out Fanconi Anemia according to PHENOS. Am. J. Med. Genet. Part A. 2016; 170(6):1520-1524.
Chaudhury S, Auerbach A D, Kernan N A, et al. Fludarabine-based cytoreductive regimen and T-cell-depleted grafts from alternative donors for the treatment of high-risk patients with Fanconi anaemia. Br. J. Haematol. 2008; 140(6):644-655.
Dufour C. How I manage patients with Fanconi anaemia. Br. J. Haematol. 2017; 178(1):32-47.
Shukla P, Ghosh K, Vundinti B R. Current and emerging therapeutic strategies for Fanconi anemia. Hugo J. 2012; 6(1):1.
Chandrakasan S, Jayavaradhan R, Ernst J, et al. KIT blockade is sufficient for donor hematopoietic stem cell engraftment in Fanconi anemia mice. Blood. 2017; 129(8):1048-1052.
Czechowicz A, Kraft D, Weissman I L, Bhattacharya D. Efficient Transplantation via Antibody-Based Clearance of Hematopoietic Stem Cell Niches. Science (80). 2007; 318(5854):1296-1299.
Chhabra A, Ring A M, Weiskopf K, et al. Hematopoietic stem cell transplantation in immunocompetent hosts without radiation or chemotherapy. Sci. Transl. Med. 2016; 8(351).
Li Z, Czechowicz A, Scheck A, Rossi D J, Murphy P M. Hematopoietic chimerism and donor-specific skin allograft tolerance after non-genotoxic CD117 antibody-drug-conjugate conditioning in MHC-mismatched allotransplantation. Nat. Commun. 2019; 10(1):616.
Czechowicz A, Palchaudhuri R, Scheck A, et al. Selective hematopoietic stem cell ablation using CD117-antibody-drug-conjugates enables safe and effective transplantation with immunity preservation. Nat. Commun. 2019; 10(1):1-12.
Agarwal R, Dvorak C C, Prohaska S, et al. Toxicity-Free Hematopoietic Stem Cell Engraftment Achieved with Anti-CD117 Monoclonal Antibody Conditioning. Biol. Blood Marrow Transplant. 2019; 25(3):S92.
Srikanthan M A, Humbert O, Haworth K G, et al. Effective Multi-lineage Engraftment in a Mouse Model of Fanconi Anemia Using Non-genotoxic Antibody-Based Conditioning. Mol. Ther.—Methods Clin. Dev. 2020; 17(June):455-464.
Reliene R, Yamamoto M L, Nagesh Rao P, Schiestl R H. Genomic instability in mice is greater in fanconi anemia caused by deficiency of Fancd2 than Fancg. Cancer Res. 2010; 70(23):9703-9710.
Xue X, Pech N K, Shelley W C, et al. Antibody targeting KIT as pretransplantation conditioning in immunocompetent mice. Blood. 2010; 116(24).
George B M, Kao K S, Kwon H S, et al. Antibody Conditioning Enables MHC-Mismatched Hematopoietic Stem Cell Transplants and Organ Graft Tolerance. Cell Stem Cell. 2019; 25(2):185-192.e3.
Wilkinson A C, Ishida R, Kikuchi M, et al. Long-term ex vivo haematopoietic-stem-cell expansion allows nonconditioned transplantation. Nature. 2019; 571(7763):117-121.

Each publication cited in this specification is hereby incorporated by reference in its entirety for all purposes.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

1. A method of hematopoietic stem cell engraftment in an individual mammal where endogenous hematopoietic stem cells (HSC) are at a growth disadvantage compared to wild-type or enhanced HSC, the method comprising:
contacting said mammal with a conditioning regimen comprising one or more immunosuppressive agents in the absence of alkylating agents or radiation, in a dose effective in achieve immunosuppression; and
introducing exogenous wild-type or enhanced HSC to said individual.

2. The method of claim 1, wherein the individual is a human.

3. The method of claim 1, wherein the individual has Fanconi Anemia (FA).

4. The method of claim 1, wherein the exogenous HSC are autologous HSC.

5. The method of claim 1, wherein the exogenous HSC are HLA-matched relative to the recipient.

6. The method of claim 1, wherein the exogenous HSC are haplo-identical or HLA-mismatched relative to the recipient.

7. The method of claim 1, wherein the exogenous HSC are enhanced through genetic modification.

8. The method of claim 1, wherein the exogenous HSC are enhanced through mRNA modification.

9. The method of claim 1, wherein the exogenous HSC are enhanced through stem cell expansion.

10. The method of claim 1, wherein the exogenous HSC are generated in vitro.

11. The method of claim 1, wherein the conditioning regimen is performed in the absence of HSC targeting antibodies.

12. The method of claim 1, wherein the immunosuppressive agents are selected from antibodies targeting one or more of CD2, CD3, CD4, CD8, CD20, CD40L, CD52, CD122, and anti-thymocyte globulin.

13. The method of claim 12, wherein a cocktail of immunosuppressive antibodies comprises antibodies specific for CD4, CD8, CD40L, and CD122.

14. The method of claim 12, wherein a cocktail of immunosuppressive antibodies comprises antibodies specific for CD4 and ATG.

15. The method of claim 1, wherein the immunosuppressive agents are antibodies targeting CD20, and anti-thymocyte globulin.

16. The method of claim 1, comprising the use of one or both of cyclophosphamide and fludarabine as immunosuppressive agents.

17. The method of claim 15, wherein the immunosuppressive agents further comprise cyclophosphamide and fludarabine.

18. The method of claim 1, wherein the immunosuppressive agents comprise rapamycin or ruxolitinib.
